(19) United States
(12) Patent Application Publication
(10) Pub. No.: US 2024/0309363 A1
(43) Pub. Date: Sep. 19, 2024

(54) COMBINATION THERAPEUTICS FOR TREATMENT OF PROLIFERATIVE DISORDERS

(71) Applicant: GEORGIA TECH RESEARCH CORPORATION, Atlanta, GA (US)

(72) Inventors: Stephen Nicholas HOUSLEY, Atlanta, GA (US); John Francis MCDONALD, Atlanta, GA (US); Minati NMI SATPATHY, Atlanta, GA (US)

(21) Appl. No.: 18/271,926

(22) PCT Filed: Jan. 12, 2022
(86) PCT No.: PCT/US2022/012129

§ 371 Date: Jul. 12, 2023

Related U.S. Application Data
(60) Provisional application No. 63/136,354, filed on Jan. 12, 2021.

(51) Int. Cl. C12N 15/11; A61K 9/06; A61K 31/506; A61K 31/517; A61K 31/519; A61K 31/5377; A61K 33/243; A61K 39/395; A61P 35/00

(52) U.S. Cl. CPC C12N 15/11; A61K 9/06; A61K 31/506; A61K 31/517; A61K 31/519; A61K 31/5377; A61K 33/243; A61K 39/3955; A61P 35/00; C12N 2310/141

(57) ABSTRACT

Disclosed herein are compositions and methods for treating proliferative disorders or sensitizing overproliferative cells to cytotoxic agents.

### CROSS-REFERENCE TO RELATED APPLICATIONS

This application claims the benefit of U.S. Provisional Application No. 63/136,354, filed on Jan. 12, 2021, which is expressly incorporated herein by reference in its entirety.

### INCORPORATION-BY-REFERENCE OF MATERIAL SUBMITTED ELECTRONICALLY

Incorporated by reference in its entirety herein is a computer-readable amino acid sequence listing submitted concurrently herewith and identified as follows: One 2.42 kilobytes ASCII (Text) file named “10034-078WO1_2022_01_12_Sequence_Listing.” created on Jan. 12, 2022.

### FIELD

The present invention relates to the field of therapeutics, and more particularly the combination of miRNAs and inhibitors of receptor tyrosine kinase (I-RTK) which together reduce epithelial-to-mesenchymal transition, induce mesenchymal-to-epithelial transition, sensitize cancer cells to I-RTK, and/or amplify the efficacy of cytotoxic agents, which is useful in the treatment of hyperproliferative diseases, such as cancers, in mammals.

### BACKGROUND

Current therapeutic approaches to hyperproliferative diseases, such as cancers, include the use of cytotoxic agents for destroying diseased cells. Side effects for cytotoxic therapies, also known as off-target effects, include nausea, pain, vomiting, hair loss, and hearing loss. Further, in some cancers, cells that do not respond to cytotoxic therapy cause the disease to recur and result in chemotherapy-resistance (Ushijima K. (2010). Treatment for recurrent ovarian cancer—at first relapse. Journal of oncology, 2010, 497429).

Greater selectivity may be obtained using proteomic or antibody matching of a chemical entity to a site on or in diseased cells or disease-causing agents, such as viruses or bacteria. Such approaches may inhibit the disease in such a way as to reduce or eliminate the disease burden.

However, many patients develop resistance, and these therapies may have off-target effects due to the protein-pocket matching of otherwise healthy tissue, limiting their efficacy (Whitebread, S., Hamon, J., Bojanic, D., & Urban, L. (2005). Keynote review: in vitro safety pharmacology profiling: an essential tool for successful drug development. Drug discovery today, 10(21), 1421-1433).

A contributing factor for resistance is cancer cells undergoing epithelial-to-mesenchymal transition (“EMT”), which plays a fundamental role in approximately 25-30% of all human cancers and promotes metastasis. Mesenchymal-like cells are slowly dividing, or even non-dividing, and are not good targets for current cytotoxic therapies, thereby contributing to failed drug trials that focus on protein level targets.

These processes are a major cause of cancer-related deaths and resistance to treatment, and, on that basis, an urgent need exists in the art to develop complementary therapeutic interventions that address the underlying disease process and avoid resistance.

### SUMMARY

The limitations of current treatment strategies can be avoided by using the compositions and methods provided herein. The compositions and methods provided herein can suppress epithelial-to-mesenchymal transition (EMT) and induce mesenchymal-to-epithelial transition (MET) to sensitize overproliferative cells (e.g., tumor cells) to the effects of inhibitors of key disease related processes. These inhibitors include genes or proteins that enhance the therapeutic effect of cytotoxic agents for the treatment of hyperproliferative diseases, such as cancer or benign hyperplasia in mammals. The methods and therapeutics described herein are based, at least in part, on the discovery that treatment with members of the miR-200 family in combination with tyrosine kinase inhibitors (e.g., receptor tyrosine kinase inhibitors). In some examples, the compositions and methods provided herein are effective in amplifying the antitumoral effects of cytotoxic agents. In some examples, the compositions and methods provided herein induce MET.

In some examples, the combination treatment disclosed herein results in significant suppression of EMT and induction of MET, via miRNAs and inhibitors of receptor tyrosine kinases (I-RTKs), and is therefore useful for the treatment of overproliferative diseases, such as cancers, in mammals. The compositions and methods disclosed herein are effective in inhibit overproliferative cells (e.g., tumor cells) from EMT and sensitizing overproliferative cells to cytotoxic therapies. Also it is shown herein that the application of EMT inhibition and gene knock-down results in a synergistic effect relative to the use of the individual techniques.

In some embodiments, tumor cells (e.g., ovarian cancer cells) that have become chemoresistant are targeted using nanoparticles loaded with a polynucleotide, e.g., a miRNA such as miRNA-429, and an EGFR inhibitor, e.g., an siRNA such as siRNA EGFR. The two mechanisms of action applied to chemoresistant tumor cells re-establish apoptosis and inhibit EMT, conferring chemo-sensitivity.

Thus, in some aspects, a composition is provided herein, said composition comprising: an miRNA selected from the group consisting of miR-200 family members; and an I-RTK. In some embodiments, the miR-200 family member is selected from the group consisting of miR-200a, miR-200b, miR-200c, miR-141, and miR-429. In some embodiments, the I-RTK is selected from the group consisting of inhibitors of EGFR, erbB2, HER3, and HER4. In some embodiments, the I-RTK is an siRNA having substantial sequence identity to a gene encoding EGFR. In some embodiments, the composition is encapsulated in a nanogel-based delivery system comprising: a nanogel comprising a crosslinked polymer particle and a crosslinked polymer shell, disposed substantially around the crosslinked polymer particle, and wherein the crosslinked polymer particle comprises poly(N-isopropylmethacrylamide) and/or N,N′-methylenebis(acrylamide).

In another aspect, a composition is provided, the composition comprising: a nanogel comprising a crosslinked polymer particle and a crosslinked polymer shell, disposed substantially around the crosslinked polymer particle; an miRNA selected from the group consisting of miR-200 family members contained substantially within the nanogel; and an siRNA having substantial sequence identity to a gene encoding EGFR contained substantially within the nanogel, wherein the miRNA and the siRNA are non-covalently associated with the nanogel, and wherein the crosslinked polymer particle comprises poly(N-isopropylmethacrylamide) and/or N,N′-methylenebis(acrylamide).

In some aspects, a method for treating cancer is provided, the method comprising administering to a mammalian subject in need thereof a composition comprising: a nanogel comprising a crosslinked polymer particle and a crosslinked polymer shell, disposed substantially around the crosslinked polymer particle; an miRNA selected from the group consisting of miR-200 family members contained substantially within the nanogel; and an siRNA having substantial sequence identity to a gene encoding EGFR contained substantially within the nanogel, wherein the miRNA and the siRNA are non-covalently associated with the nanogel, and wherein the crosslinked polymer particle comprises poly(N-isopropylmethacrylamide) and/or N,N′-methylenebis(acrylamide). In some embodiments, the composition is administered in combination with a chemotherapeutic agent.

In some aspects, use is disclosed of a composition, the composition comprising: a nanogel comprising a crosslinked polymer particle and a crosslinked polymer shell, disposed substantially around the crosslinked polymer particle; an miRNA selected from the group consisting of miR-200 family members contained substantially within the nanogel; and an siRNA having substantial sequence identity to a gene encoding EGFR contained substantially within the nanogel, wherein the miRNA and the siRNA are non-covalently associated with the nanogel, and wherein the crosslinked polymer particle comprises poly(N-isopropylmethacrylamide) and/or N,N′-methylenebis(acrylamide), in the manufacture of a medicament for the treatment of cancer. In some embodiments, the composition is administered in combination with a chemotherapeutic agent.

In some aspects, a composition, the composition comprising: a nanogel comprising a crosslinked polymer particle and a crosslinked polymer shell, disposed substantially around the crosslinked polymer particle; an miRNA selected from the group consisting of miR-200 family members contained substantially within the nanogel; and an siRNA having substantial sequence identity to a gene encoding EGFR contained substantially within the nanogel, wherein the miRNA and the siRNA are non-covalently associated with the nanogel, and wherein the crosslinked polymer particle comprises poly(N-isopropylmethacrylamide) and N, N′-methylenebis(acrylamide), is provided for use as a medicament for the treatment of cancer. In some embodiments, the composition is administered in combination with a chemotherapeutic agent.

In some aspects, a kit is provided, the kit comprising a composition, the composition comprising: a nanogel comprising a crosslinked polymer particle and a crosslinked polymer shell, disposed substantially around the crosslinked polymer particle; an miRNA selected from the group consisting of miR-200 family members contained substantially within the nanogel; and an siRNA having substantial sequence identity to a gene encoding EGFR contained substantially within the nanogel, wherein the miRNA and the siRNA are non-covalently associated with the nanogel, and wherein the crosslinked polymer particle comprises poly(N-isopropylmethacrylamide) and/or

N,N′-methylenebis(acrylamide). In some embodiments, the kit further comprises a chemotherapeutic agent.

### BRIEF DESCRIPTION OF THE DRAWINGS

FIG. 1 illustrates the anti-cancer cell effect of combination mi/siRNA treatment versus individual miRNA or siRNA treatments by graphing the percentage of viable cells remaining after further treatment with cisplatin.

FIG. 2 shows cellular images illustrating the in vitro anti-cancer cell effect of combination mi/siRNA treatment versus individual miRNA or siRNA treatments after further treatment with cisplatin.

FIG. 3 shows in vivo images of the anti-cancer cell effect of combination mi/siRNA treatment versus individual siRNA treatment after further treatment with cisplatin.

FIG. 4 graphically compares tumor weights illustrating the in vivo anti-cancer cell effect of combination mi/siRNA treatment versus individual miRNA or siRNA treatments after further treatment with cisplatin.

FIGS. 5A-5B: FIG. 5A graphically represents weight under various conditions; and FIG. 5B graphically represents the serum chemistry in control and NG groups.

FIG. 6 shows in vivo images of the anti-cancer cell effect of combination mi/siRNA treatment in different tissues of origin.

FIG. 7 represents tracing of the anti-cancer cell effect of combination mi/siRNA treatment across biologic scales.

FIGS. 8A-8B show an analysis of nanogels as used herein.

### DETAILED DESCRIPTION

Mesenchymal cells divide slowly or remain dormant. This inactivity shields mesenchymal cells from responding to therapy. By inhibiting EMT, therapies are more effective. miRNA-200 family members interact with tumor cells in a mesenchymal state, including MET, and making the cells sensitive to cancer therapeutic agents including, for example, alkylating, alkylating-like, mitotic inhibiting-, or anti-mitotic-based chemotherapeutic agents.

EMT is one of many cancer pathways inhibiting therapeutic approaches, and therefore EMT inhibition is not a complete therapeutic approach. In some examples, tumor cells that survive chemotherapy can lose the ability to undergo apoptosis, i.e., programmed cell death, by overexpressing epidermal growth factor receptor (EGFR). An additional gene knock-down approach is employed to inhibit the over-expression of EGFR by knocking down EGFR while incorporating miRNA-200 family EMT transition inhibition.

In some embodiments, tumor cells (e.g., ovarian cancer cells) that have become chemoresistant are targeted using nanoparticles loaded with a polynucleotide, e.g., a miRNA such as miRNA-429, and an EGFR inhibitor, e.g., an siRNA such as siRNA EGFR. The two mechanisms of action applied to chemoresistant tumor cells re-establish apoptosis and inhibit EMT, conferring chemo-sensitivity.

A formulation and/or composition comprising two elements, one suppressing epithelial-to-mesenchymal transition (EMT) and inducing mesenchymal-to-epithelial transition (MET) to sensitize tumor cells to the effects of inhibitors of key disease related processes, e.g., genes or proteins which ultimately enhance the therapeutic effect of cytotoxic agents, preferably members of the miR-200 family, and the second inhibiting receptor tyrosine kinases (I-RTK) (e.g., a tyrosine kinase inhibitor), causally linked to cytotoxic agent resistance, is used for the treatment of overproliferative diseases, such as cancers or benign hyperplasia, in mammals.

In some embodiments, the combination disclosed herein induces MET in tumor cells and amplifies the antitumoral effects of cytotoxic agents. This combination treatment results in significant suppression of EMT and induction of MET, via miRNAs and I-RTKs, and is therefore useful for the treatment of overproliferative diseases, such as cancers, in mammals.

Inhibiting tumor cells from transitioning from epithelial to mesenchymal sensitizes the tumor cells to cytotoxic therapies. Genetic expression inhibition has been shown to sensitize tumor cells to chemotherapy by decreasing protein and genetic expression the epidermal growth factor receptor, EGFR.

This method utilizes both a tyrosine kinase inhibitor and a miRNA in the miRNA-200 family while minimizing off-target effects and thereby reducing the required therapeutic dose required for therapeutic benefit.

In some aspects, monoclonal antibodies (mAbs) are used as an adjuvant therapy for multiple tissue origin cancers by binding extracellular domains of protein receptors to occlude ligand binding and thereby block ligand-induced activation of oncogenic pathways. mAbs can restore immunogenicity; however, dosing is limited due to increased immune response. By limiting mAbs exposure to target tissue and accompanying it with miRNA-429 for inducing MET, synergistic therapeutics effects are enabled

In some aspects, an I-RTK is administered with miRNA-429 or any other miRNA-200 family member. By intravenously delivering these therapeutics to tumor cells via targeted nanoparticles, MET is induced and tyrosine kinase activity is reduced, thereby improving the effect of chemotherapy.

The combination induces MET in cancer cells and amplifies the antitumoral effects of cytotoxic agents. This combination treatment results in significant suppression of EMT and induction of MET, via miRNAs and I-RTKs, and is therefore useful for the treatment of hyperproliferative diseases, such as cancers, in mammals.

Terms used throughout this application are to be construed with ordinary and typical meaning to those of ordinary skill in the art. However, Applicants desire that the following terms be given the particular definition as provided below.

### Definitions

As used in the specification and claims, the singular form “a.” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of +20%, +10%, +5%, or +1% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The terms “antibody” and antibodies” are used herein in a broad sense and include polyclonal antibodies, monoclonal antibodies, and bi-specific antibodies. In addition to intact immunoglobulin molecules, also included in the term “antibodies” are fragments or polymers of those immunoglobulin molecules, and human or humanized versions of immunoglobulin molecules or fragments thereof. Native antibodies are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

There are five major classes of human immunoglobulins: IgA. IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired activity.

The term “antibody fragment” refers to a portion of a full-length antibody, generally the target binding or variable region. Examples of antibody fragments include Fab, Fab′, F(ab′)₂ and Fv fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The antibody fragments, whether attached to other sequences or not, can include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition (e.g., cancer). The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term “biocompatible” generally refers to a material and any metabolites or degradation products thereof that are generally non-toxic to the recipient and do not cause significant adverse effects to the subject.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “cancer cells” and “tumor cells” are used interchangeably to refer to cells derived from a cancer or a tumor, or from a tumor cell line or a tumor cell culture.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanchisa (1984) Nucl. Acids Res. 12:203.

The term “complementary” and “complementarity” refers to the rules of Watson and Crick base pairing. For example, A (adenine) bonds with T (thymine) or U (uracil) and G (guanine) bonds with C (cytosine). For example, DNA contains an antisense strand that is complementary to its sense strand. A nucleic acid that is 95% identical to a DNA antisense strand is therefore 95% complementary to the DNA sense strand.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a proliferative disorder). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level so long as the decrease is statistically significant.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide). The term “expression vector” refers to a vector where the inserted DNA segment is operably linked to an expression control sequence.

The term “vector” refers to a replicon, such as a plasmid, phage, or cosmid, into which another DNA segment may be inserted to bring about the replication of the inserted segment.

The term “expression control sequence” refers to a DNA sequence that controls and regulates the transcription and/or translation of another DNA sequence. Control sequences that are suitable for eukaryotic cells include promoters and enhancers.”

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The terms “identical” or percent “identity.” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments;

or the end of either sequence is reached. The BLAST algorithm parameters W. T, and X determine the sensitivity and speed of the alignment. The BLASTN program (for nucleotide sequences) uses as defaults a wordlength (W) of 11, an expectation I or 10, M=5, N=−4 and a comparison of both strands. For amino acid sequences, the BLASTP program uses as defaults a wordlength of 3, and expectation I of 10, and the BLOSUM62 scoring matrix (see Henikoff and Henikoff (1989) Proc. Natl. Acad. Sci. USA 89:10915) alignments (B) of 50, expectation I of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.0.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

As used herein, “induce”, as well as the correlated term “induction”, refer to the action of generating, promoting, forming, regulating, activating, enhancing or accelerating a biological phenomenon.

Inhibit”, “inhibiting.” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 80%, optionally 50% or 25, 10%, 5% or 1%.

The term “liposome” refers to a spherical vesicle having at least one lipid layer bilayer.

MicroRNAs” (“miRNAs”) are a class of non-coding RNAs that regulate gene expression and, thereby, biological processes. miRNAs are single-stranded RNA molecules that generally range in length from about 20 to about 25 nucleotides in their naturally occurring form, although shorter and longer miRNAs have been identified. miRNAs are initially transcribed as a primary miRNA (“pri-miRNA”) that is cleaved to form one or more precursor miRNAs (“pre-miRNA”). The pre-miRNA molecule has regions of self-complementarity, forms a stem-loop structure, and is further processed by the enzyme Dicer to produce the “mature” (processed) miRNA. Complete complementary is generally not required between the mature miRNA and the target mRNA sequence; however, the “seed” region is generally less tolerant to alterations.

The term “nanoparticle” as used herein refers to a particle or structure which typically ranges from about 1 nm to about 1000 nm in size, preferably from about 50 nm to about 500 nm size, more preferably from about 50 nm to about 350 nm size, more preferably from about 100 nm to about 250 nm size.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a promoter refers to the physical and functional relationship between the DNA and promoter such that the transcription of the DNA is initiated from the promoter by an RNA polymerase that specifically recognizes, binds to, and transcribes the DNA.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods. For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z.

where W is the number of nucleotides or amino acids scored as identical matches by the sequence alignment program in that program's alignment of C and D, and where Z is the total number of nucleotides or amino acids in D. Where the length of sequence C is not equal to the length of sequence D, the % sequence identity of C to D will not equal the % sequence identity of D to C.

“Pharmaceutically acceptable” component can refer to a component that is not biologically or otherwise undesirable, i.e., the component may be incorporated into a pharmaceutical formulation disclosed herein and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21^{st} Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e., is biologically inert and physiologically acceptable, non-toxic, and is biodegradable in the environment of use, i.e. can be resorbed by the body. Examples of synthetic polymers include, but are not limited to, poly(lactic-co-glycolic acid) (PLGA). The term “polymer” encompasses all forms of polymers including, but not limited to, natural polymers, synthetic polymers, homopolymers, heteropolymers or copolymers, addition polymers, etc. The term “polymeric particle” refers to particle made out of one or more polymers.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide.” “protein,” and “polypeptide” are used interchangeably to refer to a natural or synthetic molecule comprising two or more amino acids linked by the carboxyl group of one amino acid to the alpha amino group of another.

The term “prevent” as used herein does not require absolute forestalling of the condition or disease but can also include a delay in onset or a reduction in the severity of the disease or condition. Thus, if a therapy can treat a disease in a subject having symptoms of the disease, it can also prevent that disease in a subject who has yet to suffer some or all of the symptoms.

The term “promoter” refers to an expression control sequence, typically located upstream (5′) of a DNA sequence, that, in conjunction with various elements, is responsible for regulating the transcription of the DNA sequence.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “resistance” or the like, as used herein, refers to the regression of the sensitivity to certain medicine, increment of therapeutically effective amount compared to the expected effect, after a series of course of treatment are taken by a subject in need. The term “drug-resistance” or the like, as used herein, is resistance to a single drug or multidrug resistance.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

The term “treat” or “treatment” as used herein means the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of a proliferative disorder. In some embodiments, a desired therapeutic result is the control of cancer, or a symptom of cancer. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “variant” as used herein refers to a polypeptide or polynucleotide that differs from a reference polypeptide or polynucleotide, but retains essential properties. A typical variant of a polypeptide differs in amino acid sequence from another, reference, polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions).

Unless otherwise specified, “a,” “an.” “the.” “one or more of.” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to nanogel comprising certain active ingredients, and “each nanogel” is said to have a particular active ingredient content, if there are 10 nanogels, and two or more of the nanogels have the particular active ingredient content, then that subset of two or more nanogels is intended to meet the limitation.

The term “about” in conjunction with a number is simply shorthand and is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

### II. Compositions

A. Treatments that Suppress Epithelial-to-Mesenchymal Transition (EMT) and Induce Mesenchymal-to-Epithelial Transition (MET)

In some aspects, disclosed herein is a composition comprising a miRNA in the miR-200 family and a tyrosine kinase inhibitor. Compositions include at least one miRNA that induces mRNA molecular silencing, by one or more of the following processes: (1) cleavage of the target(s) mRNA strand into pieces, (2) destabilization of the mRNA through shortening of its poly(A) tail, and/or (3) less efficient translation of the mRNA into proteins by ribosomes. In some examples, the miRNA in the miR-200 family target a number of strands. The miRNA can have hundreds of canonical targets. In some embodiments, the miRNA described herein reduces epithelial-to-mesenchymal transition and/or induces mesenchymal-to-epithelial transition, sensitizing cells to a receptor tyrosine kinases inhibitor (I-RTK) (e.g., an EGFR inhibitor).

In some embodiments, the compositions disclosed herein include at least one miRNA that induces mRNA molecular silencing, by one or more of the following processes: (1) cleavage of the target(s) mRNA strand (in this specific case, it would be a number of strands as the miRNA has hundreds of canonical targets) into two pieces; (2) destabilization of the mRNA through shortening of its poly(A) tail; and (3) less efficient translation of the mRNA into proteins by ribosomes to reduce EMT and induce MET, sensitizing cells to EGFR inhibition.

miRNAs

The term “miRNA” includes the primary (pri-miRNA), precursor (pre-miRNA), and mature forms of the miRNA. In some embodiments, the term does not include the pri-miRNA and/or pre-miRNA. The term also includes modified forms (e.g., sequence variants) of the miRNA (e.g., 1 2, 3, 4, 5, or more nucleotides that are substituted, inserted and/or deleted). In some embodiments, the variant substantially retains at least one biological activity of the wild-type miRNA. The term also includes variants that have been modified to resist degradation within a subject and/or within a cell. The term further includes fragments of a miRNA that substantially retain at least one biological activity of the wild-type miRNA. The term “substantially retains” at least one biological activity of the wild-type miRNA means at least about 50%, 60%, 70%, 80%, 90% or more of the biological activity of the wild-type miRNA is retained. The one or more biological activities of miRNA can include any relevant activity, including without limitation, binding activity (e.g., to a target mRNA), reduction or inhibition of EMT, induction of MET, prevention of metastasis, treating cancer (e.g., ovarian cancer), and/or increasing the sensitivity of a cancer cell to a cytotoxic agent.

The term “miR-200 family member” refers to an miRNA in the miR-200 family, which includes, for example, miR-200a, miR-200b, miR-200c, miR-141, and miR-429. These miRNAs are encoded in two clusters in the human genome: miR-200a, miR-200b, and miR-429 are generated as a polycistronic transcript from human chromosome 1; and miR-141 and miR-200c are generated as a single transcript from chromosome 12. Unless otherwise stated, the term also includes the primary (pri-miRNA), precursor (pre-miRNA), and mature forms of these miRNAs. The term also includes modified forms (e.g., sequence variants) of members of the miR-200 family (e.g., 1 2, 3, 4, 5, or more nucleotides that are substituted, inserted, or deleted). In some embodiments, the variant substantially retains at least one biological activity of the wild-type miRNA. The term also includes variants that have been modified to resist degradation within a subject and/or within a cell. The term further includes fragments of an miR-200 family member that substantially retain at least one biological activity of the wild-type miRNA. The term “substantially retains” at least one biological activity of the wild-type miRNA means at least about 50%, 60%, 70%, 80%, 90%, 95%, 98%, 99% or more of the biological activity of the wild-type miRNA. The one or more biological activities can include any relevant activity, including without limitation, binding activity (e.g., to a target mRNA), reduction or inhibition of EMT, induction of MET, prevention of metastasis, treating a cancer (e.g., ovarian cancer), increasing the sensitivity of a cancer cell to a cytotoxic agent.

Epithelial-mesenchymal transition (EMT) is a process that enables cancer cells to suppress their epithelial features changing to mesenchymal ones. This process allows cells to acquire mobility and the capacity to migrate from the primary site. Mesenchymal to epithelial transition (MET) is a process that converts motile mesenchymal cells to polarized epithelial cells. Methods of measuring these processes are well-known in the art, including, for example, measuring the levels of epithelial markers and mesenchymal markers. In some embodiments, cells undergoing EMT have a reduced expression of epithelial markers and an increased expression of mesenchymal markers. In some embodiments, cells undergoing MET have a reduced expression of mesenchymal markers and an increased expression of epithelial markers. In some embodiments, the epithelial makers comprise CD324/E-cadherin and/or OCLN/occludin. In some embodiments, the mesenchymal markers comprise vimentin, N-cadherin, and/or fibronectin.

In some embodiments, the miRNA disclosed herein reduces EMT of an overproliferative cell by at least about 10%, 20%, 30%, 40%, 50%, 60%, 70%, 80%, 90%, 95%, 98%, or 99% as compared to a reference control. In some embodiments, the miRNA disclosed herein improves MET of an overproliferative cell by at least about 10%, 20%, 30%, 40%, 50%, 60%, 70%, 80%, 90%, 95%, 98%, or 99% as compared to a reference control. The term “reference control” refers to a level in detected in a subject or cell in general or a study population (e.g., a non-treated subject or cell).

In some embodiments, the overproliferating cell is a human tumor cell or a cell of a hyperplastic condition. In some embodiments, the hyperplastic condition is benign hyperplasia of the skin (e.g., psoriasis or endometriosis) or prostate (e.g., benign prostatic hyperplasia).

In some embodiments, the miR-200 family member comprises the pre-miR-141, pre-miR-200a, pre-miR-200b, pre-miR-200c, and/or pre-miR-429. Representative embodiments include the human pre-miR-141 (SEQ ID NO: 7), pre-miR-200a (SEQ ID NO: 9), pre-miR-200b (SEQ ID NO: 1), pre-miR-200c (SEQ ID NO: 3), and/or pre-miR-429 (SEQ ID NO: 5), as well as mature human miR-141 (SEQ ID NO: 8), miR-200a (SEQ ID NO: 10), miR-200b (SEQ ID NO: 2), miR-200c (SEQ ID NO: 4), and/or miR-429 (SEQ ID NO: 6).

In some embodiments, the pre-miR-141 comprises a polynucleotide sequence having at or greater than about 80%, about 85%, about 90%, about 95%, or about 98% homology with SEQ ID NO: 7, or a fragment of SEQ ID NO: 7.

In some embodiments, the pre-miR-200a comprises a polynucleotide sequence having at or greater than about 80%, about 85%, about 90%, about 95%, or about 98% homology with SEQ ID NO: 9, or a fragment of SEQ ID NO: 9.

In some embodiments, the pre-miR-200b comprises a polynucleotide sequence having at or greater than about 80%, about 85%, about 90%, about 95%, or about 98% homology with SEQ ID NO: 1, or a fragment of SEQ ID NO: 1.

In some embodiments, the pre-miR-200c comprises a polynucleotide sequence having at or greater than about 80%, about 85%, about 90%, about 95%, or about 98% homology with SEQ ID NO: 3, or a fragment of SEQ ID NO: 3.

In some embodiments, the pre-miR-429 comprises a polynucleotide sequence having at or greater than about 80%, about 85%, about 90%, about 95%, or about 98% homology with SEQ ID NO: 5, or a fragment of SEQ ID NO: 5.

In some embodiments, the miR-141 comprises a polynucleotide sequence having at or greater than about 80%, about 85%, about 90%, about 95%, or about 98% homology with SEQ ID NO: 8, or a fragment of SEQ ID NO: 8.

In some embodiments, the miR-200a comprises a polynucleotide sequence having at or greater than about 80%, about 85%, about 90%, about 95%, or about 98% homology with SEQ ID NO: 10, or a fragment of SEQ ID NO: 10.

In some embodiments, the miR-200b comprises a polynucleotide sequence having at or greater than about 80%, about 85%, about 90%, about 95%, or about 98% homology with SEQ ID NO: 2, or a fragment of SEQ ID NO: 2.

In some embodiments, the miR-200c comprises a polynucleotide sequence having at or greater than about 80%, about 85%, about 90%, about 95%, or about 98% homology with SEQ ID NO: 4, or a fragment of SEQ ID NO: 4.

In some embodiments, the miR-429 comprises a polynucleotide sequence having at or greater than about 80%, about 85%, about 90%, about 95%, or about 98% homology with SEQ ID NO: 6, or a fragment of SEQ ID NO: 6.

In preferred aspects of miR-429, a modified form thereof can contain 1, 2, 3, 4, 5, or more of the following nucleotides changes, with no modifications in the seed sequence:
the “U” at position 1 of miR-429 can be replaced by an A, C and/or G and/or can be deleted.
the “U” at position 9 of miR-429 can be replaced by an A, C and/or G and/or can be deleted.
the “C” at position 10 of miR-429 can be replaced by an A, G and/or U and/or can be deleted.
the “U” at position 11 of miR-429 can be replaced by an A, C and/or G and/or can be deleted.
the “G” at position 12 of miR-429 can be replaced by an A, C and/or U and/or can be deleted.
the “G” at position 13 of miR-429 can be replaced by an A, C and/or U and/or can be deleted.
the “U” at position 14 of miR-429 can be replaced by an A, C and/or G and/or can be deleted.
The “A” at position 15 of miR-429 can be replaced by a C, G and/or U and/or can be deleted.
the “A” at position 16 of miR-429 can be replaced by a C, G and/or U and/or can be deleted.
The “A” at position 17 of miR-429 can be replaced by a C, G and/or U and/or can be deleted.
the “A” at position 18 of miR-429 can be replaced by a C, G and/or U and/or can be deleted.
the “C” at position 19 of miR-429 can be replaced by an A, G and/or U and/or can be deleted.
the “C” at position 20 of miR-429 can be replaced by an A, G and/or U and/or can be deleted.
the “G” at position 21 of miR-429 can be replaced by an A, C and/or U and/or can be deleted.
the “U” at position 22 of miR-429 can be replaced by an A, C and/or G and/or can be deleted.
In some embodiments, miR-429 comprises the polynucleotide sequence of SEQ ID NO: 6.

In some embodiments, the composition herein comprises a DNA sequence encoding the disclosed miRNA, wherein the DNA sequence is incorporated into an expression vector. In some examples, the expression vector comprising the DNA encoding the miRNA is operably linked to an expression control sequence. In some embodiments, the DNA sequence encodes a pre-miRNA sequence. In some embodiments, the DNA sequence encodes a mature miRNA sequence.

In some embodiments, miRNA is also meant to refer to those disclosed in U.S. Pat. No. 8,895,509, which is incorporated herein in its entirety.

### Inhibitors of Receptor Tyrosine Kinases (I-RTK)

The composition/formulation disclosed herein comprises an inhibitor of a tyrosine kinase inhibitor (e.g., a receptor tyrosine kinase), including, for example, a canonical inhibitor of the receptor tyrosine kinases (CI-RTKs), which uses enzymatic, proteomic, and/or genetic mechanisms to variously block, attenuate, or inhibit signal transduction. Receptor tyrosine kinases (RTKs) are cell surface receptors comprised of multimeric complexes that bind extracellular ligands to transduce transmembrane signaling that ultimately regulate key cellular processes and have been shown to play critical roles in the development and progression of many types of cancer.

In some embodiments, the receptor tyrosine kinase inhibitor provided herein can be used as antiproliferative agents for therapeutant or prophylactic treatment of a variety of human tumors (for example, renal tumor, liver tumor, kidney tumor, bladder tumor, breast tumor, gastric tumor, ovarian tumor, colorectal tumor, prostate tumor, pancreatic tumor, lung tumor, vulval tumor, thyroid tumor, hepatic carcinomas, sarcomas, glioblastomas, or various head and neck tumors), and other hyperplastic conditions such as benign hyperplasia of the skin (e.g., psoriasis or endometriosis) or prostate (e.g., benign prostatic hyperplasia (BPH)).

In some embodiments, enzymatic inhibitors of the erbB family of oncogenic and protooncogenic protein tyrosine kinases, such as EGFR, erbB2, HER3, or HER4, are used as antiproliferative agents for therapeutant or prophylactic treatment of a variety of human tumors (renal, liver, kidney, bladder, breast, gastric, ovarian, colorectal, prostate, pancreatic, lung, vulval, thyroid, hepatic carcinomas, sarcomas, glioblastomas, and various head and neck tumors) and other hyperplastic conditions, such as benign hyperplasia of the skin (e.g., psoriasis) or prostate (e.g., BPH). Examples include Gefitinib, Erlotinib, Icotinib, Afatinib, Osimertinib, and AC0010

In some embodiments, the receptor kinase inhibitor described herein is an inhibitor of the erbB family of oncogenic and protooncogenic protein tyrosine kinases. In humans, the erbB family of RTK includes EGFR (also known as HER1 or erbB1), ErbB2 (also known as HER2 or ErbB2), ErbB3 (also known as HER3), and ErbB4 (also known as HER4). In some embodiments, the EGFR protein is that identified in publicly available database as UniProtKB/Swiss-Prot: P00533-1. In some embodiments, the ErbB2 protein is that identified in publicly available database as UniProtKB/Swiss-Prot: P04626-1. In some embodiments, the ErbB3 protein is that identified in publicly available database as UniProtKB/Swiss-Prot: P21860-1. In some embodiments, the ErbB4 protein is protein is that identified in publicly available database as UniProtKB/Swiss-Prot: Q15303-1.

In some embodiments, the tyrosine kinase inhibitor is selected from the group consisting of a small molecule, a protein, and a nucleic acid. In some embodiments, the tyrosine kinase inhibitor is selected from gefitinib, erlotinib, icotinib, afatinib, osimertinib, and AC0010.

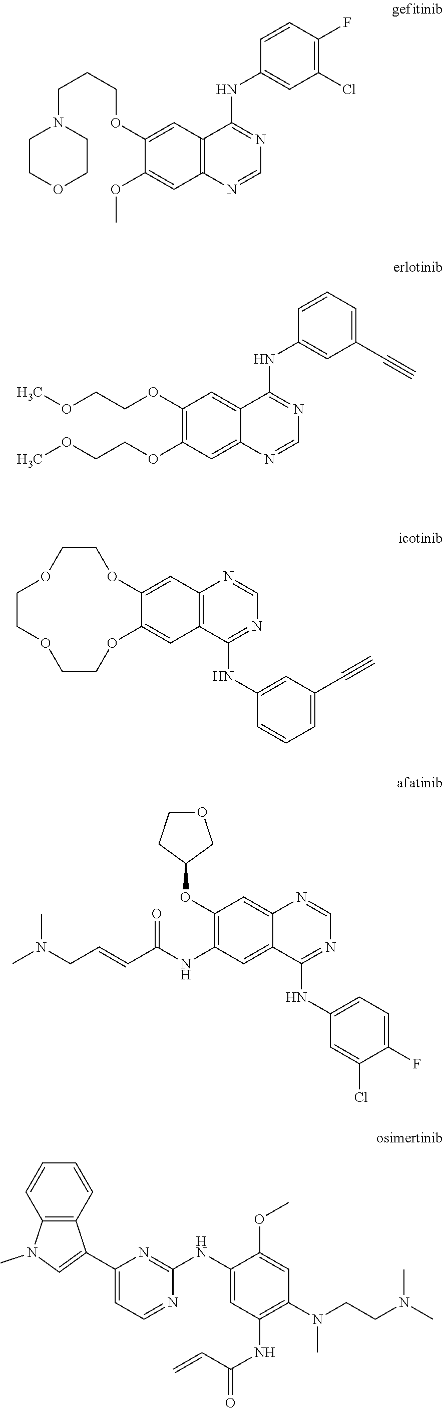

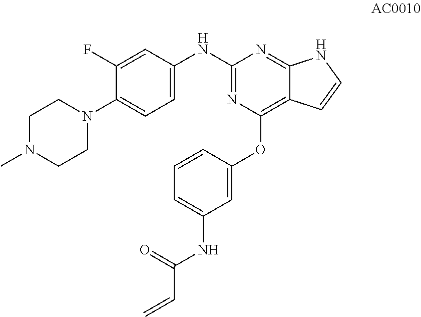 [SMILES: C=CC(=O)Nc1cccc(Oc2nc(Nc3ccc(N4CCN(C)CC4)c(F)c3)nc3[nH]ccc23)c1]

In some embodiment, the inhibitor is a protein. In some embodiments, the inhibitor is a glycoprotein. The term “glycoprotein” herein refers to amino acid sequences that include one or more covalently attached oligosaccharide chains (e.g., glycans). Example glycoproteins include glycosylated antibodies and antibody-like molecules (e.g., Fc fusion proteins). Example antibodies include monoclonal antibodies and/or fragments thereof, polyclonal antibodies and/or fragments thereof, and Fc domain containing fusion proteins (e.g., fusion proteins containing the Fc region of IgG1, or a glycosylated portion thereof). A glycoprotein preparation is a composition or mixture that includes at least one glycoprotein. Example monoclonal antibodies include: cetuximab, panitumumab, nimotuzumab, and necitumumab.

In some embodiments, the inhibitor is a small interfering RNA (siRNA). As used herein, the terms “small interfering RNA,” “siRNA,” “interfering RNA”, “RNAi”, or “interfering RNA sequence” refers to double-stranded RNA (i.e., duplex RNA) that silences, reduces, or inhibits the expression of a target gene (e.g., by mediating the degradation of mRNAs which are complementary to the sequence of the interfering RNA) when the interfering RNA is in the same cell as the target gene.

siRNA refers to the double stranded RNA formed by two complementary strands or by a single, self-complementary strand (e.g., a hairpin). The siRNA typically has substantial or complete identity to the target gene. The sequence of the siRNA can correspond to the full-length target gene, or a sub-sequence (i.e., a portion) thereof. siRNA includes interfering an RNA of about 15 to about 60 nucleotides in length, about 15 to about 50 nucleotides in length, or about 15 to about 40 nucleotides in length, more typically about 15 to about 30 nucleotides in length, 15 to about 25 nucleotides in length, or 19 to about 25 nucleotides in length, and is preferably about 21 to about 25 nucleotides in length, about 20 to about 24 nucleotides in length, about 21 to about 22 nucleotides in length, or about 21 to about 23 nucleotides in length. In some embodiments, the siRNA duplex comprises a 3′ overhang and 5′ phosphate termini, wherein the 3′ overhang is about 1 to about 4 nucleotides in length, preferably of about 2 to about 3 nucleotides in length. The siRNA can be chemically synthesized or can be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli Rnase III or Dicer. These enzymes process the dsRNA into biologically active siRNA. In some embodiments, the dsRNA is at least 50 nucleotides to about 100, 200, 300, 400, or 500 nucleotides in length. In some embodiments, the dsRNA is about 1000, 1500, 2000, 5000 nucleotides in length, or longer. In some embodiments, the siRNA used herein targets a EGFP polynucleotide.

As used herein, siRNA can target (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA typically has substantial or complete identity to the target gene. The sequence of the siRNA can correspond to the full-length target gene or a sub-sequence (i.e., a portion) thereof. siRNA includes interfering RNA of about 15 to about 60 nucleotides, about 15 to about 50 nucleotides, or about 15 to about 40 nucleotides in length, more typically about 15 to about 30 nucleotides, 15 to about 25 nucleotides, or 19 to about 25 nucleotides. In some aspects, siRNA includes interfering RNA of about 21 to about 25 nucleotides, about 20 to about 24 nucleotides, about 21 to about 22 nucleotides, or about 21 to about 23 nucleotides. siRNA duplexes may comprise 3′ overhangs of about 1 to about 4 nucleotides, including about 2 to about 3 nucleotides, and 5′ phosphate termini.

The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA. In one aspect, dsRNA are at least 50 nucleotides to about 100, 200, 300, 400, or 500 nucleotides in length. A dsRNA may be as long as 1000, 1500, 2000, or 5000 nucleotides in length, or longer. The dsRNA can encode for an entire gene transcript or a partial gene transcript.

In some embodiments, the siRNA targeting EGFR used herein comprises a sequence having at or greater than about 80%, about 85%, about 90%, about 95%, or about 98% homology with SEQ ID NO: 11 or 12, or a fragment of SEQ ID NO: 11 or 12.

In some embodiments, siRNA is also meant to refer to those disclosed in U.S. Pat. No. 8,361,510, which is incorporated herein in its entirety.

In some embodiments, the composition further comprises a therapeutically effective amount of a cytotoxic agent.

As used herein, the term “cytotoxic agent” or “chemotherapeutic agent” refers to a substance that kills cancer cells and/or stops cancer cells from dividing and growing. The cytotoxic agent can cause tumors to shrink in size. The cytotoxic agent can be used for chemotherapy. In some embodiments, the cytotoxic agent is selected from cisplatin, carboplatin, and paclitaxel.

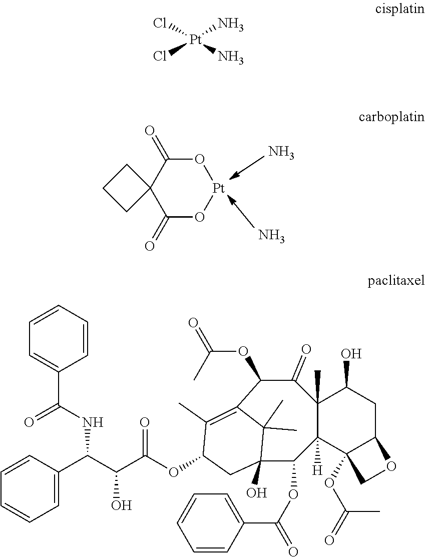

In some embodiments, the composition or formulation disclosed herein targets a tissue associated with a proliferative disorder. In some embodiments, the composition is in an amount effective to reduce epithelial-to-mesenchymal transition and/or induce mesenchymal-to-epithelial transition of one or more overproliferating cells in the tissue, thereby sensitizing the one or more overproliferating cells to a receptor tyrosine kinases inhibitor (I-RTK) and/or amplifying the efficacy of cytotoxic agents in reducing proliferation or viability of the one or more overproliferating cells.

In some embodiments, the composition disclosed herein reduces EMT of an overproliferative cell by at least about 10%, 20%, 30%, 40%, 50%, 60%, 70%, 80%, 90%, 95%, 98%, or 99% as compared to a reference control. In some embodiments, the composition disclosed herein improves MET of an overproliferative cell by at least about 10%, 20%, 30%, 40%, 50%, 60%, 70%, 80%, 90%, 95%, 98%, or 99% as compared to a reference control. The term “reference control” refers to a level in detected in a subject or cell in general or a study population (e.g., a non-treated subject or cell).

### IV. Excipients

By “pharmaceutically acceptable” it is meant a material that is not biologically or otherwise undesirable, i.e., the material can be administered to a subject without causing any undesirable biological effects such as toxicity.

The formulations can optionally comprise medicinal agents, pharmaceutical agents, carriers, adjuvants, dispersing agents, and diluents.

The miRNAs (or vectors encoding miRNAs) and one or more of the receptor tyrosine kinase inhibitors (e.g., CI-RTKs) can be formulated for administration in a pharmaceutical carrier in accordance with known techniques. The miRNA or vector (including the physiologically acceptable salts thereof) and one or more of the receptor tyrosine kinase inhibitors (e.g., CI-RTKs) are typically admixed with an acceptable carrier. The carrier can be a solid, a liquid, or both, and can be formulated with the miRNA and the receptor tyrosine kinase inhibitors (e.g., CI-RTKs) as a unit-dose formulation, for example, a tablet, which can contain from 0.01 or 0.5% to 95% or 99% by weight of the miRNA and the receptor tyrosine kinase inhibitors (e.g., CI-RTKs) or vectors. One or more miRNAs and the receptor tyrosine kinase inhibitors (e.g., CI-RTKs) or vectors can be incorporated in the formulations, which can be prepared by any of the well-known techniques of pharmacy.

Non-limiting examples of formulations include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor).

The most suitable route in any given case will depend on the nature and severity of the condition being treated and on the nature of the compound being used. In some embodiments, the formulation can be delivered locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be a slow-release formulation, e.g., in the form of a slow-release depot.

In some embodiments, the pharmaceutically acceptable carrier is a polymeric particle or a liposome. In some embodiments, the pharmaceutically acceptable carrier is a nanoparticle.

### III. Methods for Making the Compositions

An miRNA can be constructed using chemical synthesis and enzymatic ligation reactions by procedures known in the art. For example, an miRNA can be chemically synthesized using naturally occurring nucleotides or various modified nucleotides designed to increase the biological stability of the molecules or to increase the physical stability of the duplex formed between the miRNA and target nucleotide sequences, e.g., phosphorothioate derivatives and acridine substituted nucleotides can be used. Examples of modified nucleotides that can be used to generate the miRNA include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

Alternatively, the miRNA can be produced using an expression vector into which a nucleic acid encoding the miRNA has been cloned. Methods to construct expression vectors containing genetic sequences and appropriate transcriptional and translational control elements are well known in the art. These methods include in vitro recombinant DNA techniques, synthetic techniques, and in vivo genetic recombination. Vectors include but are not limited to plasmids, viral nucleic acids, viruses, phage nucleic acids, phages, cosmids, and artificial chromosomes.

In some aspects, the vector is derived from either a virus or a retrovirus. Viral vectors include adenovirus, adeno-associated virus, herpes virus, vaccinia virus, polio virus, HIV virus, neuronal trophic virus, Sindbis, and other viruses. Also useful are any viral families that share the properties of these viruses that make them suitable for use as vectors. Typically, viral vectors contain nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed, and a gene or gene/promoter cassette is inserted into the viral genome in place of the removed viral DNA. The necessary functions of the removed early genes are typically supplied by cell lines that have been engineered to express the gene products of the early genes in trans.

Expression vectors generally contain regulatory sequences, which are necessary elements for the translation and/or transcription of the inserted coding sequence. For example, the coding sequence can be operably linked to a promoter and/or enhancer to control the expression of the desired gene product.

Selection of the promoter to express the gene of interest will depend on the vector, the nucleic acid cassette, the cell type to be targeted, and the desired biological effect. Selection parameters can include: achieving sufficiently high levels of gene expression to achieve a physiological effect; maintaining a critical level of gene expression; achieving temporal regulation of gene expression; achieving cell type specific expression; achieving pharmacological, endocrine, paracrine, or autocrine regulation of gene expression; and preventing inappropriate or undesirable levels of expression. Any given set of selection requirements will depend on the conditions but can be readily determined once the specific requirements are determined.

Promoters can generally be divided into constitutive promoters, tissue-specific or development-stage-specific promoters, inducible promoters, and synthetic promoters. Constitutive promoters direct expression in virtually all tissues and are largely, if not entirely, independent of environmental and developmental factors. As their expression is normally not conditioned by endogenous factors, constitutive promoters are usually active across species and even across kingdoms. A promoter of this type is the CMV promoter (650 bases).

Tissue-specific or development-stage-specific promoters direct the expression of a gene in specific tissue(s) or at certain stages of development.

The performance of inducible promoters is not conditioned to endogenous factors but to environmental conditions and external stimuli that can be artificially controlled. Within this group, there are promoters modulated by abiotic factors such as light, oxygen levels, heat, cold, and wounding. Since some of these factors are difficult to control outside an experimental setting, promoters that respond to chemical compounds, not found naturally in the organism of interest, are of particular interest.

An enhancer is a sequence of DNA that functions at no fixed distance from the transcription start site and can be either 5′ or 3′ to the transcription unit. Furthermore, enhancers can be within the coding sequence itself. They are usually between 10 and 300 bp in length, and they function in cis. Enhancers function to increase transcription from nearby promoters. Enhancers also often contain response elements that mediate the regulation of transcription. Enhancers often determine the regulation of expression of a gene. While many enhancer sequences are now known from mammalian genes (e.g., globin, elastase, albumin, α-fetoprotein and insulin), typically one will use an enhancer from a eukaryotic cell virus for general expression. Preferred examples are the SV40 enhancer on the late side of the replication origin (e.g., bp 100-270), the cytomegalovirus early promoter enhancer, the polyoma enhancer on the late side of the replication origin, and adenovirus enhancers.

The promotor and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

### IV. Methods for Using the Compositions

In some aspects, disclosed herein is a method of treating a proliferative disorder in a subject in need thereof, comprising administering a therapeutically effective amount of the composition provided herein, wherein the composition comprises a miRNA in the miR-200 family and a tyrosine kinase inhibitor.

It is herein contemplated that the term “proliferative disorder” or “proliferative disease” refers to a disorder or disease characterized by uncontrolled and excessive growth of cells. In some embodiments, the proliferative disorder is a cancer. In some embodiments, the cancer is ovary cancer.

In some aspects, disclosed herein is a method of treating a proliferative disorder in a subject in need thereof, comprising administering a therapeutically effective amount of a miRNA in the miR-200 family and a therapeutically effective amount of a tyrosine kinase inhibitor.

Also disclosed herein is a method of sensitizing one or more overproliferative cells in a subject to a cytotoxic agent, comprising administering to the subject a therapeutically effective amount of a composition comprising a miRNA in the miR-200 family and a tyrosine kinase inhibitor. In some embodiments, the method further comprising administering to the subject a therapeutically effective amount of a cytotoxic agent. In some embodiments, the composition reduces epithelial-to-mesenchymal transition and/or induces mesenchymal-to-epithelial transition of the one or more overproliferating cells, thereby sensitizing the one or more overproliferating cells to a receptor tyrosine kinases inhibitor (I-RTK). In some embodiments, the overproliferative cells are tumor cells.

In some aspects, disclosed herein in a method of sensitizing one or more tumor cells in a subject to a cytotoxic agent, comprising administering to the subject a therapeutically effective amount of a composition comprising a miRNA in the miR-200 family and a tyrosine kinase inhibitor. In some embodiments, the method further comprising administering to the subject a therapeutically effective amount of a cytotoxic agent. In some embodiments, the composition reduces epithelial-to-mesenchymal transition and/or induces mesenchymal-to-epithelial transition of the one or more tumor cells, thereby sensitizing the one or more overproliferating cells to a receptor tyrosine kinases inhibitor (I-RTK).

In some examples, the amounts of a cytotoxic agent in the composition and method disclosed herein can be generally smaller, e.g., at least about 10% smaller, than the amount of the cytotoxic agent present in the current dosage of the treatment regimen (i.e., without in combination with the composition disclosed herein) required for producing essentially the same therapeutic effect. Stated another way, combining the cytotoxic agent and the composition disclosed herein can increase its therapeutic efficacy, i.e., a smaller amount of cytotoxic agent as compared to the amount present in a typical one dosage administered for cancer treatment (e.g., ovarian cancer treatment), can achieve essentially the same therapeutic effect. For example, if the traditionally recommended dosage of a cytotoxic agent (e.g., cisplatin) is X amount then the methods described herein can comprise the cytotoxic agent (e.g., cisplatin) in an amount of about 0.9×, about 0.8×, about 0.7×, about 0.6×, about 0.5×, about 0.4×, about 0.3×, about 0.2×, about 0.1× or less. Low-dosage administration of the cytotoxic agent (e.g., cisplatin) can reduce side effects of the cytotoxic agent (e.g., cisplatin), if any, and/or reduce likelihood of the subject's resistance to the cytotoxic agent (e.g., cisplatin) after administration for a period of time.

In some embodiments, the dosing frequency of the cytotoxic agent (e.g., cisplatin) in the methods disclosed herein is less (e.g., about 2-fold less, about 3-fold less, about 4-fold less, about 5-fold less, about 6-fold less, about 7-fold less, about 8-fold less, about 9-fold less, about 10-fold less, about 15-fold less, about 20-fold less, about 30-fold less, about 40-fold less, or about 50-fold less) than the dosing frequency of the cytotoxic agent (e.g., cisplatin) when the cytotoxic agent (e.g., cisplatin) is administered without the combination treatment of composition disclosed herein.

In some embodiments, monoclonal antibodies (mAbs) are used as an adjuvant therapy for multiple tissue origin cancers by binding extracellular domains of protein receptors to occlude ligand-binding and thereby block ligand-induced activation of oncogenic pathways. mAbs can restore immunogenicity, however, the dosing is limited due to increased immune response. By limiting mAbs exposure to target tissue and accompanying it with miRNA-429 for inducing MET, synergistic therapeutics effects are enabled.

In another embodiment, a tyrosine kinase inhibitor is administered with miRNA-429 or any other miRNA-200 family member. By delivering these therapeutics to tumor cells via targeted nanoparticles intravenously MET is induced and tyrosine kinase activity is reduced, thereby improving the effect of chemotherapy.

The receptor tyrosine kinase inhibitor can be administered prior to, concurrently with, and/or after administration of the miR-200 family member to a subject. By “concurrently” it is meant that the two treatments are sufficiently close in time to have a combined effect. The one or more miR-200 family members are delivered to the subject concurrently with and/or within about 3 hours to about 24, 48, 72, 96, 120, 144 or 168 hours prior to administration of one or more of the receptor tyrosine kinase inhibitors, within about 6 hours to about 24, 48, 72, 96, 120, 144 or 168 hours prior to administration of one or more of the receptor tyrosine kinase inhibitors, within about 12 hours to about 24, 48, 72, 96, 120, 144 or 168 hours prior to administration of one or more of the receptor tyrosine kinase inhibitors, within about 24 hours to about 48, 72, 96, 120, 144 or 168 hours prior to administration of one or more of the receptor tyrosine kinase inhibitors, within about 36 hours to about 72, 96, 120, 144 or 168 hours prior to administration of one or more of the receptor tyrosine kinase inhibitors, within about 48 hours to about 72, 96, 120, 144 or 168 hours prior to administration of one or more of the receptor tyrosine kinase inhibitors, or within about 72 hours to about 96, 120, 144 or 168 hours prior to administration of one or more of the receptor tyrosine kinase inhibitors.

In some embodiments, the method disclosed herein further comprises administering to the subject a therapeutically effective amount of a chemotherapeutic agent. A chemotherapeutic agent can be administered prior to, concurrently with and/or after administration of the one or more miR-200 family members and CI-RTKs, to a subject. By “concurrently” it is meant that the treatments are sufficiently close in time to have a combined effect. In some embodiments, one or more miR-200 family members and CI-RTKs delivered to the subject concurrently with and/or within about 3 hours to about 24, 48, 72, 96, 120, 144 or 168 hours prior to administration of a cytotoxic therapy, within about 6 hours to about 24, 48, 72, 96, 120, 144 or 168 hours prior to administration of a cytotoxic treatment, within about 12 hours to about 24, 48, 72, 96, 120, 144 or 168 hours prior to administration of a cytotoxic therapy, within about 24 hours to about 48, 72, 96, 120, 144 or 168 hours prior to administration of a cytotoxic therapy, within about 36 hours to about 72, 96, 120, 144 or 168 hours prior to administration of a cytotoxic therapy, within about 48 hours to about 72, 96, 120, 144 or 168 hours prior to administration of a cytotoxic therapy, or within about 72 hours to about 96, 120, 144 or 168 hours prior to administration of a cytotoxic therapy.

### Dosing

A “therapeutically effective amount” or “an effective amount” is any quantity of the active agent, which, when administered to a subject, causes prevention, reduction, remission, regression, or elimination of a neoplastic-related pathology. For example, in the context of cancer, “an effective amount” is considered to be any quantity of the one or more active agents, which, when administered to a subject, causes prevention, reduction, remission, regression, or elimination of tumorigenesis and/or metastasis.

In some aspects, the compounds are administered to the subject in a treatment effective amount, as that term is defined herein. Dosages of pharmaceutically active compounds can be determined by methods known in the art, see, e.g., Remington's Pharmaceutical Sciences (Maack Publishing Co., Easton, Pa.). The therapeutically effective dosage of any specific compound will vary somewhat from compound to compound, and patient to patient, and will depend upon the condition of the patient and the route of delivery. As a general proposition dosages from 0.001 μg/kg/day to about 1,000 mg/kg/day may be used depending on the route of administration, the active agent administered and the toxicity. More specifically, a dosage from about 0.1 to about 50 mg/kg is expected to have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

### VARIOUS EMBODIMENTS

Compositions are provided for use as a medicament for the treatment of cancer, the compositions comprising: a nanogel comprising a crosslinked polymer particle and a crosslinked polymer shell, disposed substantially around the crosslinked polymer particle; an miRNA selected from the group consisting of miR-200 family members contained substantially within the nanogel; and an siRNA having substantial sequence identity to a gene encoding EGFR contained substantially within the nanogel, wherein the miRNA and the siRNA are non-covalently associated with the nanogel, and wherein the crosslinked polymer particle comprises poly(N-isopropylmethacrylamide) and N,N′-methylenebis(acrylamide). In some aspects, the compositions are administered in combination with a chemotherapeutic agent.
1. A composition, comprising:
a micro-ribonucleic acid (an “miRNA”) selected from the group consisting of miR-200 family members; and
an inhibitor of receptor tyrosine kinases (an “I-RTK”).
2. The composition of claim 1, wherein the miR-200 family member is selected from the group consisting of miR-200a, miR-200b, miR-200c, miR-141, and miR-429.
3. The composition of any of the preceding claims, wherein the miR-200 family member is miR-429.
4. The composition of any of the preceding claims, wherein the I-RTK is selected from the group consisting of inhibitors of epidermal growth factor receptor (“EGFR”), erbB2, HER3, and HER4.
5. The composition of any of the preceding claims, wherein the I-RTK is an inhibitor for EGFR.
6. The composition of any of the preceding claims, wherein the I-RTK is a small interfering ribonucleic acid (an “siRNA”) having substantial sequence identity to a gene encoding EGFR.
7. The composition of any of the preceding claims, wherein the composition is encapsulated in a nanogel-based delivery system comprising: a nanogel comprising a crosslinked polymer particle and a crosslinked polymer shell, disposed substantially around the crosslinked polymer particle, and wherein the crosslinked polymer particle comprises poly(N-isopropylmethacrylamide) and N,N′-methylenebis(acrylamide).
8. A composition, comprising:
a nanogel comprising a crosslinked polymer particle and a crosslinked polymer shell, disposed substantially around the crosslinked polymer particle;
a micro-ribonucleic acid (an “miRNA”) selected from the group consisting of miR-200 family members contained substantially within the nanogel; and
a small interfering ribonucleic acid (an “siRNA”) having substantial sequence identity to a gene encoding epidermal growth factor receptor (“EGFR”) contained substantially within the nanogel,
wherein the miRNA and the siRNA are non-covalently associated with the nanogel, and
wherein the crosslinked polymer particle comprises poly(N-isopropylmethacrylamide) and N,N′-methylenebis(acrylamide).
9. The composition of claim 8, wherein the miR-200 family member is selected from the group consisting of miR-200a, miR-200b, miR-200c, miR-141, and miR-429.
10. The composition of any of preceding claim 8 or 9, wherein the miR-200 family member is miR-429.
11. The composition of any of preceding claims 8-10, wherein the I-RTK is selected from the group consisting of inhibitors of epidermal growth factor receptor (“EGFR”), erbB2, HER3, and HER4.
12. The composition of any of preceding claims 8-11, wherein the I-RTK is an inhibitor for EGFR.
13. The composition of any of preceding claims 8-12, wherein the I-RTK is a small interfering ribonucleic acid (an “siRNA”) having substantial sequence identity to a gene encoding EGFR.
14. A method for treating cancer, the method comprising administering to a mammalian subject in need thereof a composition, the composition comprising:
a nanogel comprising a crosslinked polymer particle and a crosslinked polymer shell, disposed substantially around the crosslinked polymer particle;
an miRNA selected from the group consisting of miR-200 family members contained substantially within the nanogel; and
an siRNA having substantial sequence identity to a gene encoding EGFR contained substantially within the nanogel,
wherein the miRNA and the siRNA are non-covalently associated with the nanogel, and
wherein the crosslinked polymer particle comprises poly(N-isopropylmethacrylamide) and N,N′-methylenebis(acrylamide).
15. The method of claim 14, wherein the composition is administered in combination with a chemotherapeutic agent.
16. The method of any of preceding claim 14 or 15, wherein the miR-200 family member is selected from the group consisting of miR-200a, miR-200b, miR-200c, miR-141, and miR-429.
17. The method of any of preceding claims 14-16, wherein the miR-200 family member is miR-429.
18. The method of any of preceding claims 14-17, wherein the I-RTK is selected from the group consisting of inhibitors of epidermal growth factor receptor (“EGFR”), erbB2, HER3, and HER4.
19. The method of any of preceding claims 14-18, wherein the I-RTK is an inhibitor for EGFR.
20. The method of any of preceding claims 14-19, wherein the I-RTK is a small interfering ribonucleic acid (an “siRNA”) having substantial sequence identity to a gene encoding EGFR.
21. Use of a composition, the composition comprising:
a nanogel comprising a crosslinked polymer particle and a crosslinked polymer shell, disposed substantially around the crosslinked polymer particle;
an miRNA selected from the group consisting of miR-200 family members contained substantially within the nanogel; and
an siRNA having substantial sequence identity to a gene encoding EGFR contained substantially within the nanogel,
wherein the miRNA and the siRNA are non-covalently associated with the nanogel, and
   wherein the crosslinked polymer particle comprises poly(N-isopropylmethacrylamide) and N,N′-methylenebis(acrylamide), in the manufacture of a medicament for the treatment of cancer.
22. The use of claim 21, wherein the composition is administered in combination with a chemotherapeutic agent.
23. The use of any of preceding claim 21 or 22, wherein the miR-200 family member is selected from the group consisting of miR-200a, miR-200b, miR-200c, miR-141, and miR-429.
24. The use of any of preceding claims 21-23, wherein the miR-200 family member is miR-429.
25. The use of any of preceding claims 21-24, wherein the I-RTK is selected from the group consisting of inhibitors of epidermal growth factor receptor (“EGFR”), erbB2, HER3, and HER4.
26. The use of any of preceding claims 21-25, wherein the I-RTK is an inhibitor for EGFR.
27. The use of any of preceding claims 21-26, wherein the I-RTK is a small interfering ribonucleic acid (an “siRNA”) having substantial sequence identity to a gene encoding EGFR.
28. A composition, the composition comprising:
a nanogel comprising a crosslinked polymer particle and a crosslinked polymer shell, disposed substantially around the crosslinked polymer particle;
an miRNA selected from the group consisting of miR-200 family members contained substantially within the nanogel; and
an siRNA having substantial sequence identity to a gene encoding EGFR contained substantially within the nanogel,
wherein the miRNA and the siRNA are non-covalently associated with the nanogel, and
wherein the crosslinked polymer particle comprises poly(N-isopropylmethacrylamide) and N,N′-methylenebis(acrylamide), for use as a medicament for the treatment of cancer.
29. The composition of claim 28, wherein the use includes administration in combination with a chemotherapeutic agent.
30. The composition of any of preceding claim 28 or 29, wherein the miR-200 family member is selected from the group consisting of miR-200a, miR-200b, miR-200c, miR-141, and miR-429.
31. The composition of any of preceding claims 28-30, wherein the miR-200 family member is miR-429.
32. The composition of any of preceding claims 28-31, wherein the I-RTK is selected from the group consisting of inhibitors of epidermal growth factor receptor (“EGFR”), erbB2, HER3, and HER4.
33. The composition of any of preceding claims 28-32, wherein the I-RTK is an inhibitor for EGFR.
34. The composition of any of preceding claims 28-33, wherein the I-RTK is a small interfering ribonucleic acid (an “siRNA”) having substantial sequence identity to a gene encoding EGFR.
35. A kit, the kit comprising a composition, the composition comprising:
a nanogel comprising a crosslinked polymer particle and a crosslinked polymer shell, disposed substantially around the crosslinked polymer particle;
an miRNA selected from the group consisting of miR-200 family members contained substantially within the nanogel; and
an siRNA having substantial sequence identity to a gene encoding EGFR contained substantially within the nanogel,
wherein the miRNA and the siRNA are non-covalently associated with the nanogel, and
wherein the crosslinked polymer particle comprises poly(N-isopropylmethacrylamide) and N,N′-methylenebis(acrylamide).
36. The kit of claim 35, further comprising a chemotherapeutic agent.
37. The kit of any of preceding claim 35 or 36, wherein the miR-200 family member is selected from the group consisting of miR-200a, miR-200b, miR-200c, miR-141, and miR-429.
38. The kit of any of preceding claims 35-37, wherein the miR-200 family member is miR-429.
39. The kit of any of preceding claims 35-38, wherein the I-RTK is selected from the group consisting of inhibitors of epidermal growth factor receptor (“EGFR”), erbB2, HER3, and HER4.
40. The kit of any of preceding claims 35-39, wherein the I-RTK is an inhibitor for EGFR.
41. The kit of any of preceding claims 35-40, wherein the I-RTK is a small interfering ribonucleic acid (an “siRNA”) having substantial sequence identity to a gene encoding EGFR.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claim.

### Example 1. Combinatorial Treatment of miR-429 and siRNA Against EGFR Amplifies Sensitivity of HEY Cells to Cisplatin In Vitro

### Materials and Methods

### Cell Lines.

The OVCAR3 cell line was obtained from the American Type Culture Collection (Manassas, Va.). The HEY cell line was kindly provided by Gordon Mills, Department of Molecular Therapeutics, University of Texas, MD Anderson Cancer Center.

microRNA and siRNA Transfection.

6×10⁴ cells per well were seeded in 24-well plates. After 24 h, cells were transfected with 30 nM of miR-429 and miR-320 miRNA oligonucleotides (Ambion, Austin, Tex.) and siRNA against EGFR (Thermo Fisher) using Lipofectamine 2000 reagent (Invitrogen, Carlsbad, Calif.). The Ambion Pre-miRNA Precursor Negative Control was used as a control. Three days after transfection, cells were split and transfected again. This process was repeated every 3 days until 21 days when cells were assayed for response.

### Cell Viability and Antitumor Activity

Hey or SK-OV-3 cells were plated in 96-well cell culture plates at a concentration of 1×10⁴ cells/well. Hey or SK-OV-3 cells were subjected to nanogel delivery of siRNA at nanogel concentrations of 1000, 100, 10, and 1 μg/mL. Forty-eight hours after siRNA and miRNA delivery, cisplatin was added to Hey or SK-OV-3 cells at concentrations ranging from 0.625-10 uM. Treatment wells were set up in four replicates, and the cells were incubated with cisplatin for an additional 4 days. After treatment, the cells were washed with PBS, and 100 μL of medium was added back to the wells. To this, 10 μL of Tox8 was added to determine cell viability. The cells were incubated with the Tox8 reagent according to the manufacturer's instructions. The fluorescence was measured (λem=560 nm, λex=590 nm) by a Spectramax Gemini Fluorescence Microplate Reader (Molecular Devices, Sunnyvale, Calif.). Wells without cells but with Tox8 were used as controls and subtracted from all treatments as background. Each experiment was performed in duplicate.

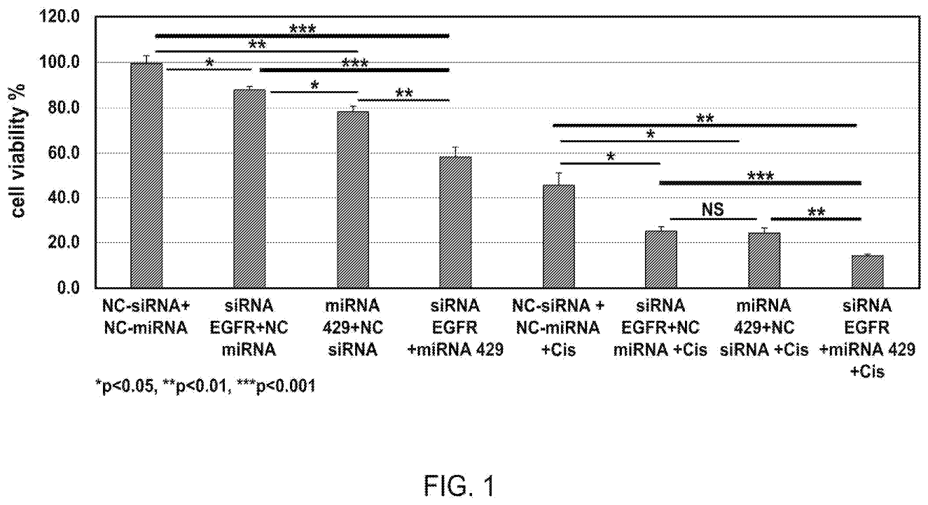

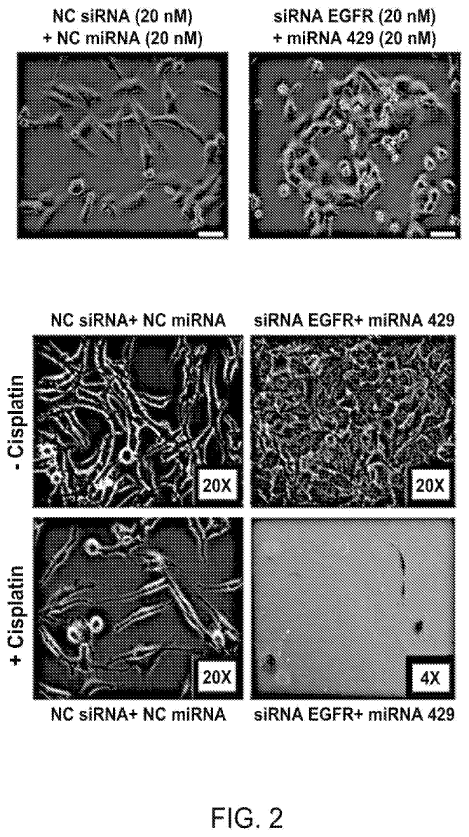

Cultures of the mesenchymal HEY cells were transfected with a negative control (“NC”) miRNA or miR-429 and (NC) siRNA or siRNA-EGFR using Lipofectamine 2000 (Invitrogen) and treated with increasing concentrations (0.625, 1.2, 2.5, 5, 10 μM) of Cisplatin. Sensitivity to cisplatin increased significantly in the presence of both miR-429 and siRNA-EGFR as compared with the negative control and displayed synergistic decrease in cell viability compared to either miR-429 or siRNA-EGFR treatment alone. Results are graphically represented in FIG. 1, and cellular images are shown in FIG. 2.

### Example 2. In Vivo Synergy of miR-429 and siRNA Therapeutic Treatment Amplifies Sensitivity to Cisplatin

### Animal Models

Five to six-week-old female severe combined immunodeficiency (SCID) mice (NOD.CB17-Prkdscid/NcrCrl, strain code 394) were purchased from Charles River Laboratories. The mice were housed and maintained under pathogen-free conditions in facilities approved by the American Association for Accreditation of Laboratory Animal Care and in accordance with current regulations and standards of the U.S. Department of Agriculture, U.S. Department of Health and Human Services, and NIH. All studies were approved by the Georgia Institute of Technology Institutional Animal Care and Use Committee. For acute toxicity study (dose-escalation), five to six-week-old female treatment naïve CD-1 mice are used.

### Tumor Induction

### Ovarian Cancer

Hey A8-F8 ovarian cancer cells 5×10⁶ were injected intraperitoneally (i.p.) into the NOD/SCID mice. For non-invasive cell imaging, D-luciferin (150 mg/kg) was administered orally to mice restrained by the scruff method. The volume of injected cells-used for study was 500 μL. The volume of injected D-luciferin was 150-200 μL.

Intraperitoneal injections of tumor cells (one time per animal) and D-luciferin (150 mg/kg) for tumor cell imaging was administered to caudal right abdominal quadrant in unanesthetized mice restrained by the scruff method. After tumor establishment within a week time, luciferin was injected intraperitoneally, and bioluminescence imaging was used to measure the tumor growth.

### Colorectal Cancer

SW480-Luc2 colorectal cancer cells 5×10⁶ were injected intraperitoneally (i.p.) into the NOD/SCID mice. For non-invasive cell imaging, D-luciferin (150 mg/kg) was administered orally to mice restrained by the scruff method. The volume of injected cells used for the study was 500 μL. The volume of injected D-luciferin was 150-200 μL.

### In Vivo Imaging

The SCID.NOD mice (6 to 8 weeks old) were placed under anesthesia either by ketamine cocktail (via IP injection) or by isoflurane for bioluminescence imaging (BLI). Mice bearing the HeyA8-F8 luciferase gene positive tumors received an i.p. injection of D-luciferin, a substrate for firefly luciferase, followed by BLI using in vivo imaging system (IVIS spectrum Ct from PerkinElmer, located at IBB animal facility) within a week after injection of tumor cells. Once a BLI signal was detectable (˜7-10 days), siRNA against EGFR was delivered intravenously and erlotinib was administered orally to different groups. After imaging, animals were placed on a heated pad and continuously monitored until their recovery. For each imaging session, the animal was sedated by anesthesia for not more than 30 minutes. Before and after each imaging session, work surfaces and equipment (imaging chamber) were disinfected using a paper towel soaked with alcohol. Bioluminescence imaging on mice was performed no more than 2 times per week. Mice were imaged up to 6 weeks and tumor progression for untreated group and regression for therapy group were monitored weekly during the therapy. For quantification of luminescence signal, the region of interest (ROI) was selected by encircling the tumor area as well as the body background from displayed images using IVIS software. Simultaneous evaluation of nano gel distribution was achieved with fluorescence in vivo imaging.

Finally, integrated flux of photons (total flux p/s) in each region was calculated by using inbuilt IVIS Caliper software, and total flux p/s value of body background was subtracted from tumor intensity (total flux p/s) every week to follow the longitudinal tumor progression as well as tumor regression in the case of treated mice.

### Assessment of Tumor Burden

Tumor burden was assessed by body weight, body condition scoring, and ascites monitoring, as well as bioluminescence signal rendered by the luciferase positive Hey A8-F8 cells or SW480-Luc2 used for the development of the IP ovarian or colorectal mouse model, respectively.

Body weight was determined 2 times per week in all mice and daily in animals displaying evidence of ascites formation (abdominal expansion, etc.). Only low to moderate ascites formation was observed and only in “control” (tumor growth without treatment) in prior experiments. Any animals presenting evidence of extensive abdominal distension, i.e., enough to cause discomfort or interfere with normal activity, were euthanized.

Body condition scoring by Ullman-Cullere was determined 2 times per week and daily in animals showing evidence of developing ascites. If animals appeared to display evidence of significant ascites formation, they were euthanized. In those animals, tumor induced body weight loss (tumor cachexia) can be masked by ascites related increase in body weight. For this reason, body condition score 1 indicating emaciation was implemented as an immediate euthanasia criterion regardless of animal body weight. Body condition scoring by Ullman-Cullere and Foltz (Laboratory Animal Science 1999; 49: 319-323) was found to be reliable indicator of well-being even in animals with ascites and organ enlargements. These scores were determined by visual examination and palpation of vertebrae and dorsal pelvis. Since formation of ascites may impair the ability to determine body condition scores from visual inspection, greater emphasis was given to examination by palpation.

Because of the use of an intra-peritoneal ovarian/colorectal cancer mouse model, the measurable tumor outside the body of the mouse was not able to be seen. Based on previous IVIS bioluminescence imaging results with the IP mouse tumor model (in NOD SCID mouse; DOI: 10.1038/srep36518), the expected tumor size for advanced staged tumor (3 weeks after the administration of the 3-5 million tumor cells) in terms of total flux is ˜6.01E+09 photons/sec or average radiance ˜2.65E+07 in terms of photons/sec/cm2/sr.

Animals were evaluated for body weight not less than 2 times/week and 7 days per week including weekends and holidays for toxicity studies, and if any animal showed signs of developing ascites, its body mass was monitored daily. The formation of ascites was determined visually by monitoring abdominal distension. Animals displaying evidence of excessive ascites formation (highly extended abdomens resulting in impaired mobility, etc.) were euthanized.

### Nanogel Synthesis

The synthesis of the nanogel will be described in more detail in Example 3. Briefly, nanogels were synthesized via emulsion precipitation polymerization. In a typical core nanogel synthesis, 0.772 g N-isopropylmethacrylamide (NIPMAm) monomer, 0.0.0191 g N,N′-methylene-bis(acrylamide) (BIS) cross linker, and 0.1135 g sodium dodecyl sulfate (SDS) surfactant were dissolved in 49 ml water. The solution was stirred under nitrogen. After the solution was brought to 70° C. under nitrogen, 0.1 mM acrylamido-fluorescein (AFA) co-monomer and 0.5 ml of a 0.01 g/ml aqueous ammonium persulfate (APS) solution were added to initiate polymerization. The polymerization was carried out at 70° C. for 4 hours under nitrogen.

The core nanogels were used as seed for the addition of a shell layer. Briefly, a 50 mM monomer solution with molar ratios of 97.5% NIPMAm, 2% BIS, and 0.5% aminopropyl methacrylate (APMA, Poly sciences) was prepared in 39.5 mL of dH2O. When the temperature stabilized at 70° C., the reaction was initiated by adding a 0.5 mL aliquot of 0.05 M APS. The reaction proceeded for 4 hours under N₂ gas. After cooling down to room temperature, core-shell particles were filtered through a 0.2 μm filter, and the size of the core-shell nanogel is measured by dynamic light scattering (DLS) equipment (Zetasizer Nano-ZS S-90).

Loading of Nanogels with siRNA or miRNA

The nanogels were loaded with siRNA and/or miRNA using the “breathing in” method. Briefly, 4 mg of lyophilized nanogel was rehydrated in 250 μl of siRNA EGFR (Life Technologies Corporation, Cat #4390825) and/or miRNA429 or negative control mi/siRNA (Life Technologies Corporation Cat #4390843) (20 μM stock made in sterile PBS) for 2 hours with rotation at 4° C. After the siRNA and/or miRNA were encapsulated in the nanogels, they were centrifuged and resuspended to a final concentration of 10 mg/mL in reduced serum media (OPTI-MEM, Thermo Fisher Scientific). The final concentration of siRNA was typically 16-17 μg siRNA/miRNA/mg of nanogels.

Injections of siRNA/miRNA in Nanogels

For tail vein injections, animals were placed in plastic restraint device and their tails were warmed by immersion in warm water ˜37° C. until optimal dilation of the veins was achieved. After disinfecting the injection site with alternating chlorhexidine and isopropyl alcohol swabs, a 27-gauge or smaller needle was inserted about 3 mm into the vein in the middle part of the tail.

### Administration of Cisplatin

Cisplatin was dissolved in 0.9% saline (obtained from PRL) for one time use and administered intraperitoneally (IP). Animals were removed gently from the cage and were restrained appropriately in the head-down position. Anatomical landmarks were identified and disinfected with 70% alcohol swab to inject into the appropriate area of the abdomen. The injection site was in the animal's lower right quadrant of the abdomen to avoid damage to the urinary bladder, cecum, and other abdominal organs. Cisplatin was warmed to room temperature since injection of cold substances can cause discomfort and drop in body temperature. By using a 25-27 g needle, cisplatin (up to 100 μL) was injected to the mouse as per the doses mentioned in the protocol.

The needle was inserted with bevel facing “up” into the lower right quadrant of the abdomen toward the head at a 30-40° angle to horizontal. The needle was inserted to the depth in which the entire bevel is within the abdominal cavity. The needle was pulled straight out. Each animal was injected with a fresh needle. Finally, the animals were placed back into their cages and monitored for few minutes for any complications.

### Oral Administration of Erlotinib (Alternative RTK Inhibitor)

One handed manual restraint of the mouse was followed for oral gavage procedure. The maximum dosing volume was 10 ml/kg used for the mouse. A sterile, disposable oral gavage needle, 18-20 gauge, was used, depending on the age/weight of the mouse. The needle tip was introduced and run along the upper palate mouth until the esophagus was reached.

### Study Design

### Single Dose Efficacy

A single dose efficacy was undertaken to evaluate the antitumoral effects of single (siRNA-EGFR or miRNA-429) or dual agent (siRNA-EGFR and miRNA-429) therapy when combined with cisplatin. The dosing schedule followed the below generalized schedule:

### Timeline

Mice received and acclimatized for a minimum of 3 days;

Day 0-tumor cells injection, IP;

Day 8+/−3 days-anesthesia for imaging 20-30 min for each mouse, and recovery luciferin injection;

Randomization to group membership;

Group siRNA+miRNA+cisplatin=“Active”;

Group siRNA+nc-miRNA+cisplatin=“siRNA”;

Group nc-siRNA+miRNA+cisplatin=“miRNA”;

Group nc-siRNA+nc-miRNA+cisplatin=“Passive”;

Group control=“Control”;

Days 8+/−4 days-group membership treatment of active miRNA (tail vein iv injection) or nc-miRNA;

Days 9+/−4 days-group membership treatment of active siRNA (tail vein iv injection) or nc-siRNA;

Day 10+/−4 days-Cisplatin treatment intraperitoneally (IP) on the following day; Next 11 Days-anesthesia for imaging 20-30 min for each mouse and recovery;

Day 19+/−4 days-terminal anesthesia for imaging, euthanasia, tissue collection (defined below).

### Acute Toxicity Evaluation

CD-1 mice were injected with group membership treatment in a dose-escalating manner and sacrificed either 7 or 14 days after treatment initiation to study the acute toxicity of single doses. The doses for nanogel loaded siRNA EGFR were determined in a dose escalating manner with a starting dose at 1 mg/kg to 14 mg/kg (1, 3.5, 7.0, 10, 14 mg/kg).

Each dose was given to at least 5 mice before the next higher dose was given. If more than one mouse met euthanasia criteria, the maximum tolerated dose (MTD) was determined and no higher doses were given. Based on maximum tolerated dose and acute toxicity study, repeated dose toxicity was conducted on CD-1 female mice by using 5 different doses (low, mod, efficacy, high, and high+), as well as a no treatment control group . . .

### Biodistribution of Nanogels

Nanogel distribution and retention was monitored via clinical grade near-infrared fluorescent (NIRF) probe, chemically (permanently) bonded to the nanogels during synthesis. Further, NIRF probes were permanently bound to nanogels. NIRF imaging occurred simultaneously with BLI imaging. NIRF biodistribution studies took part in all experimental groups utilizing nanogels; however, the independent toxicology profile of the nanogels was completed with vehicle group that contained blank nanogels.

### Endpoints

Mice were euthanized if any one of the following occurred: 1. The Ullman-Cullere body score was 1, indicating emaciation (Ullman-Culleré M H, Foltz C J. Body condition scoring: a rapid and accurate method for assessing health status in mice. Lab Anim Sci. 1999 June; 49(3): 319-23). 2. Body weight loss of about 10% or weight gain attributable to ascites equaled 10-12 g. (10-12 g of body weight increase in 6-8 week old mice attributable to ascites was observed in a study approved by the Institutional Review Board of the Wistar Institute and the University of Pennsylvania (Zhang L, Yang N, Garcia J R, et al., Am J Pathol. 2002 December; 161(6):2295-309). This represents in the animals studied about 40% body weight increases from their basal weight. 3. Distress with signs of hunching or abdominal posture or labored breathing or vocalization. 4. Moribund/lethargic animals. 5. Any condition interfering with eating or drinking. Mice were euthanized with CO2 (with a flow rate of 30-70% chamber volume/minute) following IP anesthesia with ketamine 30-65 mg/kg/xylazine (6-13 mg/kg)/acepromazine (1-2 mg/kg).

### Tissue Collections

The tumors and other tissues were collected and either frozen or fixed in PFA for further analysis. The tumors were processed for histological analysis and blood was collected to measure the toxicity levels in plasma.

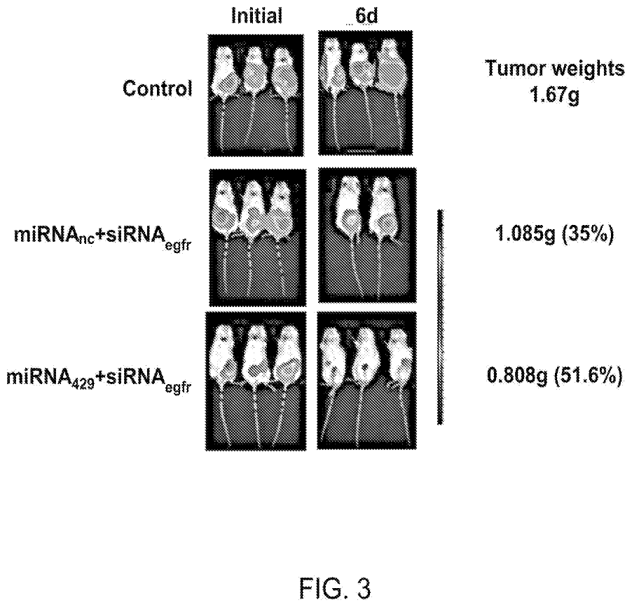

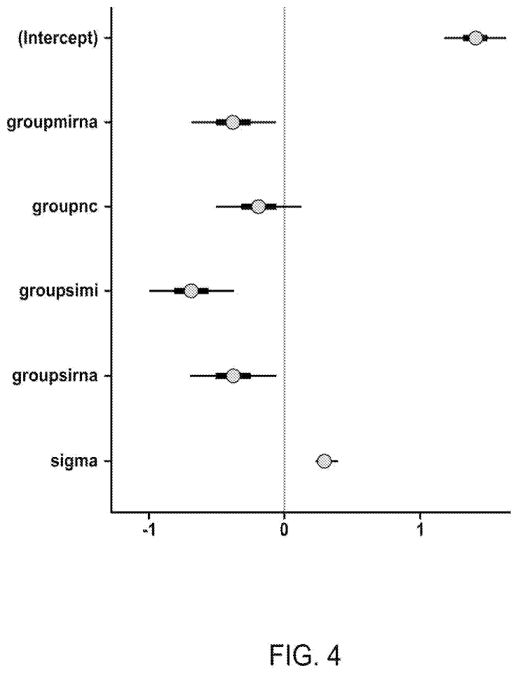

Referring now to FIGS. 3 and 4, significant reduction in tumor weight was demonstrated in animals to which a combination of miRNA and siRNA was administered.

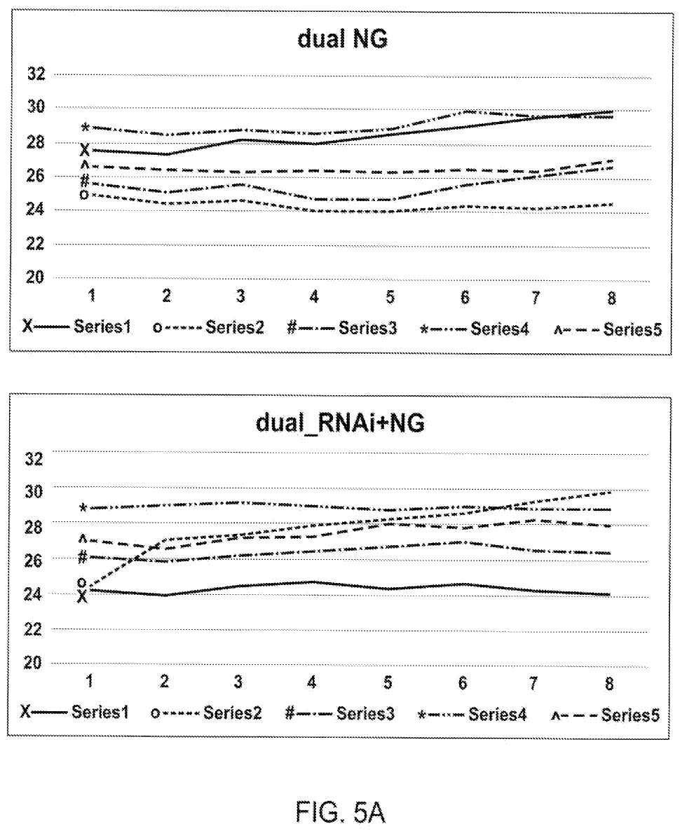

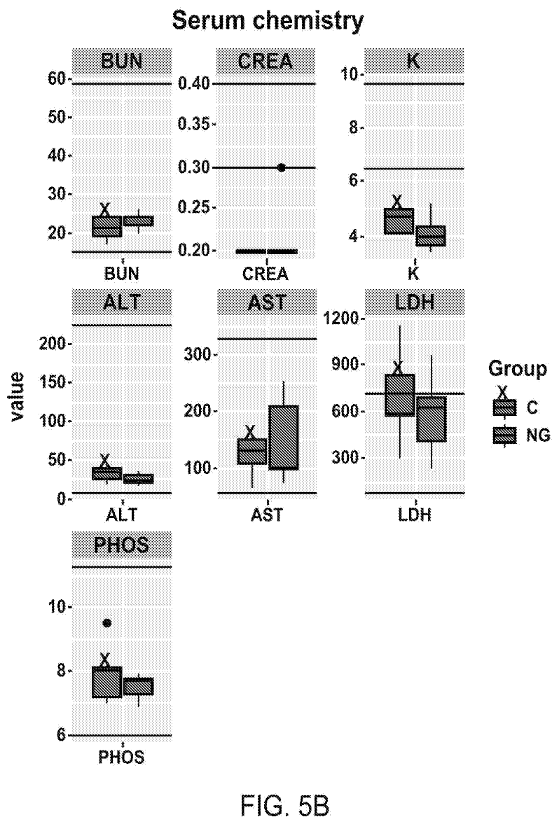

FIG. 5 addresses the toxicity of the therapeutic as administered.

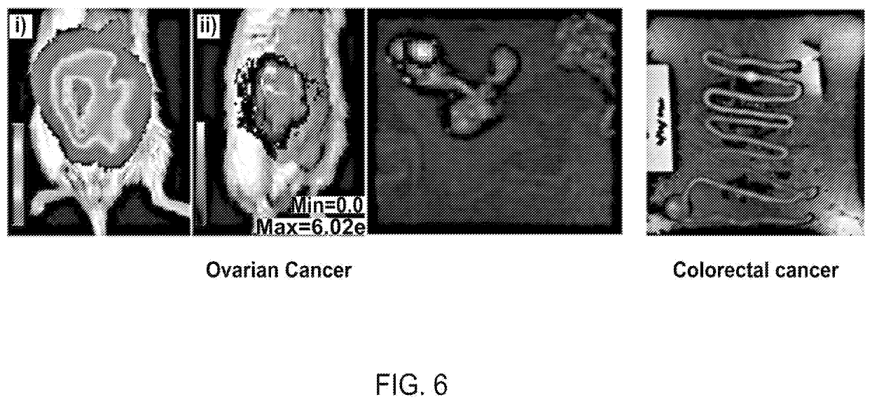

FIG. 6 demonstrates the novel targeting of the therapeutics to a tissue of origin.

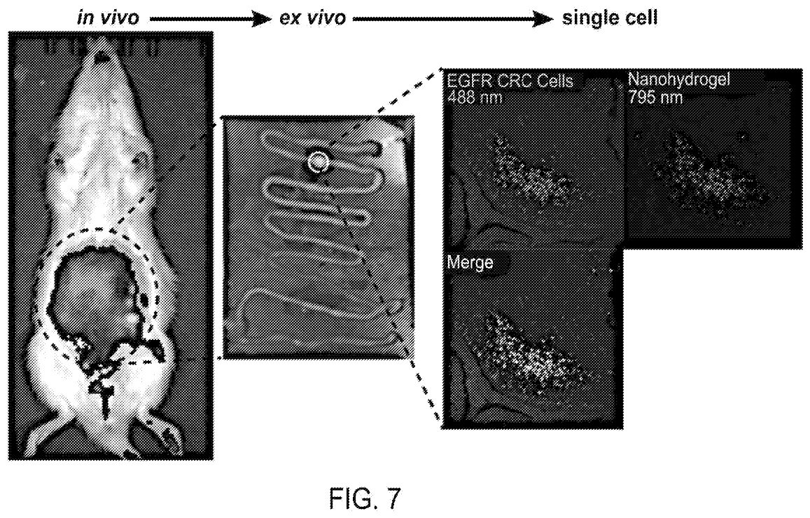

FIG. 7 demonstrates that the therapeutics according to various aspects of the invention were able to trace cancerous cells across biologic scales.

### Example 3. Nanogel Core Particle Formation, Example siRNA and miRNA Delivery Vehicle

Nanogel core particles were synthesized by free-radical precipitation polymerization. The use of thermally phase separating polymers enables the use of precipitation polymerization for the synthesis of highly monodispersed nanogels. The core particle synthesis steps were as follows:

### Day 1—Core Particle Synthesis

1. a) Poly(N-isopropylmethacrylamide) (NIPMAm) 117.8 mM (monomer) b) N,N′-methylenebis(acrylamide) 6.2 mM (cross linker) c) Sodium dodecyl sulfate (SDS) 8 mM (surfactant).
2. Put the above chemicals directly in a dark bottle, add 49.5 mL deionized (DI) water, shake it on a shaker with mild rotation for 15 mins to make a homogenous solution. Measure the volume of the solution in a graduated cylinder and adjust the volume to 49.5 ml with DI water.
3. Use 0.8 μm filter (Pall corporation) and 30 ml syringe to filter the core solution to another dark bottle.
4. Pour the solution to a 100 mL round bottom flask.
5. Put the round bottom flask in a mineral oil bath and heat the solution to 70° C. with vigorous stirring, making sure the water circulation is on for the condensation. When the temperature of the solution reaches 60° C., purge N2 (adjust N2 flow@ 1.45ssc-1.6 ssc) gas inside the solution).
6. Let the temperature of the solution stabilize at 70° C.
7. Add 200 ul of DMSO to the round bottom flask by removing N2 needle.
8. After 10 mins, add 0.5 ml of freshly prepared 800 mM APS (Ammonium persulfate) dropwise (prepare 1.0 ml 800 mM stock in DI water, (182.5 mg APS/ml DI water-800 mM), filter through 0.2 μm) by removing N2 gas needle.
9. Maintain thermal stability of the solution for 4 h.
10. Within 10-20 mins, observe a sign of turbidity in the solution, which portrays the initiation of polymerization.
11. After 4 h, stop the heater, let the particle cool down to room temperature, and turn off the N2 gas and water circulation.
12. Filter the solution through Whatman 3 mm filter paper and store at 4° C. in a dark bottle.

1st check point—Measure the size of the core particles by DLS. If the radius size is 50-55 nm, proceed with shell synthesis by using this core particle.

The core nanogels described above were used as seeds for the addition of a hydrogel shell in a seeded precipitation polymerization scheme. The shell particle synthesis steps are as follows:

### Day 2—Shell Particle Synthesis

1. Take 20 ml core particle, add 115.4 mgs SDS (4 mM) in a 3 neck round bottom flask. Put the flask inside the mineral oil bath.
2. Heat the solution with stirring under N₂ blanket, condensate water.
3. In a separate container (a total 50 mM monomer), add NIPMAm (489.8 mgs/48.75 mM), BIS acrylamide (12.173 mgs/1 mM), and APMA (2.3 mgs/0.25 mM) to the dark bottle, add 79 ml DI water. Shake for ˜15 min to get a homogeneous solution.
4. Filter the shell solution into another dark bottle, then carefully add to the three-neck round bottom flask (250 ml capacity flask for 100 ml core-shell particle synthesis) by opening the N2 gas stopper.
5. Heat the solution to 70° C. with vigorous stirring, under N₂ blanket, and condensate water.
6. When temperature reaches 60° C., purge N2 inside the solution. Allow the solution to be stabilized at 70° C. for an hour.
7. Once temperature is stabilized, add 0.5 mL of 0.05M (114 mgs in 1 mL DI water and filter) APS into the solution.
8. Observe initiation of polymerization and thermal stability of the solution for 4 h.
9. After 4 h, stop heating, let the particle cool down to room temperature, and turn off the N₂.
10. Filter the solution, measure the volume and save it in a dark bottle at 4° C. (core-shell Lot #1-100 mL).
11. Repeat the synthesis of core-shell particle Lot #2, 100 mL.

### Purification

### Day 3-4 Purification

1. Add ˜26 ml of core-shell particles into each polycarbonate ultracentrifuge tube-8 tubes. Total=158 ml.
2. Use Beckman XPN Optima-100 floor ultracentrifuge to wash these particles by using 60,000 RPM at 22° C. for 3× for 3 h each, each time removing the supernatant and re-suspending in 25 ml DI water, vortex, and resuspend the particle evenly.
3. In the last round of ultra-centrifugation, remove the supernatant, re-suspend in 3-5 ml DI water in each tube, and vortex it to mix it completely. Take glass tubes, weigh without cap, then equally distribute the purified nanogel suspension in DI water in the tubes. Tighten the cap and seal the cap with parafilm.

### Day 4-7 Freeze Drying of the Particles

1. Prepare the acetone-dry ice bath. In a 250 ml beaker, pour half of acetone inside the hood. Keep adding the dry ice into the acetone. Dip the glass tube containing nanogel suspension into the dry ice-acetone solution until it is completely frozen. Once it is completely frozen, remove parafilm, loosen the cap, put the glass vials inside the lyophilization flask, and lyophilize the nanogels for 72 h.
2. After 72 h, weigh the glass vial again without cap to get the weight of the particles and save the dry particles inside the refrigerator.
3. Test each batch for endotoxin before injecting into animals. The preferred lyophilization setting for 72 h is under a vacuum of about 0.049 mbar at a temperature of about −51.1° C.

### Recrystallization of NIPMAM-Hexane Recrystallization

Recrystallization is a technique used to purify solid compounds. The rate of cooling determines the size and quality of the crystals: rapid cooling favors small crystals; and slow cooling favors the growth of large and generally purer crystals. Place 25 g NIPMAm in an Erlenmeyer flask. Add 1 liter Hexane to a 2 L Erlenmeyer flask. Put the flask with a magnetic bar over a digital hotplate with stirrer. Adjust the temperature to 70° C. (set 3) to get the temp of the solution to 43° C. and keep stirring (level 7) until NIPMAm goes into solution except some traces of inhibitors. Let it cool down at room temperature. Store at 4° C. for overnight to form crystals. Use vacuum pump with ceramic funnel covered with Whatman 1 mm paper. Pour the chilled crystals on the ceramic funnel and wash the flask with extra chilled hexane. Take out the dry crystals and put on the ceramic plate inside the hood and cover with a paper towel. Leave it for 2-3 days inside the hood.

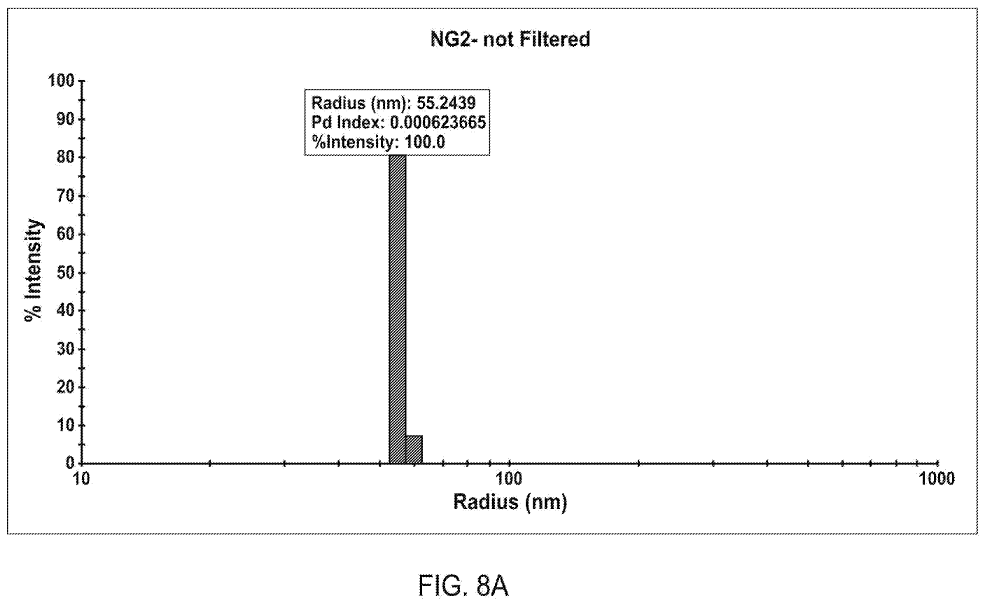

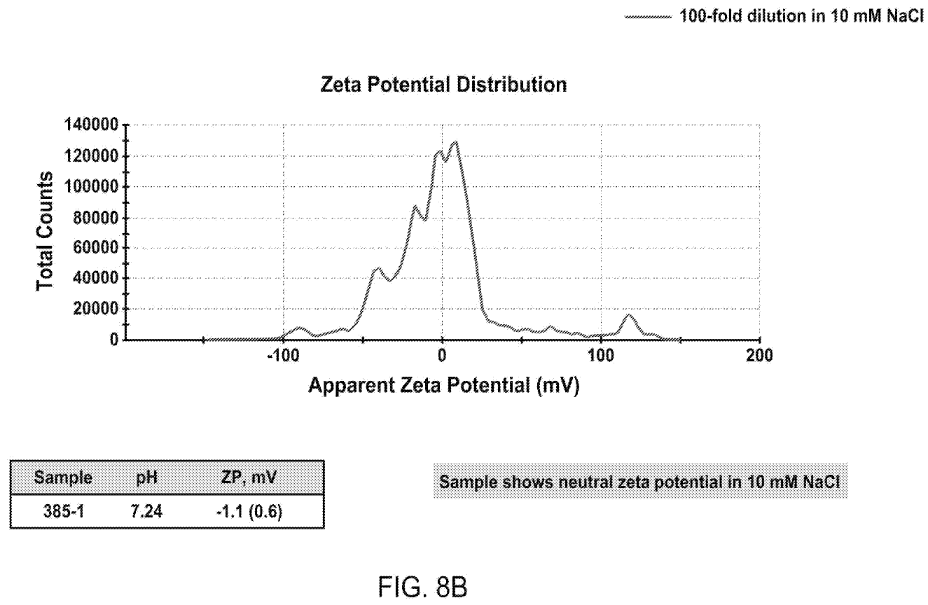

Nanogel sample preparation for particle size by DLS: The core-shell nanogel powder is resuspended in PBS. pH 7.4 at the concentration of 1 mg/ml. For hydrodynamic size, 1 or 0.5 mg/ml core-shell nanogel solution is used, whereas for zeta potential, 0.1-0.3 mg/ml core-shell nanogel solution is used to avoid any possible particle aggregation (FIGS. 8A-8B).

The typical size of the nanogel varies from 56-62 nm+4 nm (D₅₀) (@ 1 mg/ml in PBS, pH 7.4)-(Wyatt DynaPro NanoStar Dynamic Light Scattering instrument used).

A suitable nanogel delivery system is described in U.S. Pat. No. 8,361,510, which is incorporated herein by reference in its entirety.

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

All patents, patent applications, and publications referenced herein are incorporated by reference in their entirety for all purposes.

Human miR-200 family member sequences

| hsa-miR- | | |
|---|---|---|
| 200 | | Mature miRNA |
| Family | | (with seed |
| Member | Precursor miRNA | region in bold) |
| miR-200b | CCAGCUCGGGCAGCCGUGGCC | UAAUACUGCCU |
| | AUCUUACUGGGCAGCAUUGGA | GGUAAUGAUGA |
| | UGGAGUCAGGUCUCUAAUACU | (SEQ ID |
| | GCCUGGUAAUGAUGACGGCGG | NO: 2) |
| | AGCCCUGCACG | |
| | (SEQ ID NO: 1) | |
| miR-200c | CCCUCGUCUUACCCAGCAGUG | UAAUACUGCCGG |
| | UUUGGGUGCGGUUGGGAGUCU | GUAAUGAUGGA |
| | CUAAUACUGCCGGGUAAUGAU | (SEQ ID |
| | GGAGG | NO: 4) |
| | (SEQ ID NO: 3) | |
| miR-429 | CGCCGGCCGAUGGGCGUCUUA | UAAUACUGUCU |
| | CCAGACAUGGUUAGACCUGGC | GGUAAAACCGU |
| | CCUCUGUCUAAUACUGUCUGG | (SEQ ID |
| | UAAAACCGUCCAUCCGCUGC | NO: 6) |
| | (SEQ ID NO: 5) | |
| miR-141 | CGGCCGGCCCUGGGUCCAUCU | UAACACUGUCU |
| | UCCAGUACAGUGUUGGAUGGU | GGUAAAGAUGG |
| | CUAAUUGUGAAGCUCCUAACA | (SEQ ID |
| | CUGUCUGGUAAAGAUGGCUCC | NO: 8) |
| | CGGGUGGGUUC | |
| | (SEQ ID NO: 7) | |
| miR-200a | CCGGGCCCCUGUGAGCAUCUU | UAACACUGUCU |
| | ACCGGACAGUGCUGGAUUUCC | GGUAACGAUGU |
| | CAGCUUGACUCUAACACUGUC | (SEQ ID |
| | UGGUAACGAUGUUCAAAGGUG | NO: 10) |
| | ACCCGC | |
| | (SEQ ID NO: 9) | |

Sequence for siRNA EGFR

Sense (5′-3′)
(SEQ ID NO: 11)
CCAUAAAUGCUACGAAUAUTT
Anti-sense(5′-3′)
(SEQ ID NO: 12)
AUAUUCGUAGCAUUUAUGGAG

1. A composition comprising:
a miRNA in the miR-200 family, and
a tyrosine kinase inhibitor.

2. The composition of claim 1, wherein the miRNA in the miR-200 family is selected from the group consisting of miR-200a, miR-200b, miR-200c, miR-141, and miR-429.

3. The composition of claim 1, wherein the tyrosine kinase inhibitor is selected from the group consisting of a small molecule, a protein, and a nucleic acid.

4. The composition of claim 3, wherein the small molecule has a molecular weight of under 1500 Daltons.

5. The composition of claim 1, wherein the tyrosine kinase inhibitor inhibits one or more members of the erbB family of oncogenic and protooncogenic protein tyrosine kinases.

6. The composition of claim 1, wherein the tyrosine kinase inhibitor is selected from the group consisting of an epidermal growth factor receptor (EGFR) inhibitor, an erbB2 inhibitor, a HER3 inhibitor, and a HER4 inhibitor.

7. The composition of claim 1, wherein the tyrosine kinase inhibitor is selected from the group consisting of gefitinib, erlotinib, icotinib, afatinib, osimertinib, and AC0010.

8. The composition of claim 3, wherein the tyrosine kinase inhibitor is an antibody or a fragment thereof.

9. The composition of claim 8, wherein the antibody is a humanized antibody.

10. The composition of claim 1, further comprising a pharmaceutically acceptable carrier.

11. The composition of claim 10, wherein the pharmaceutically acceptable carrier is a liposome or a nanogel.

12. The composition of claim 11, wherein the nanogel comprises a crosslinked polymer particle and a crosslinked polymer shell.

13. The composition of claim 12, wherein the crosslinked polymer particle comprises poly(N-isopropylmethacrylamide) and N,N′-methylenebis(acrylamide).

14. The composition of claim 1, wherein the composition targets a tissue associated with a proliferative disorder.

15. The composition of claim 14, wherein the proliferative disorder is a cancer.

16. The composition of claim 14, wherein the composition reduces epithelial-to-mesenchymal transition and/or induces mesenchymal-to-epithelial transition of one or more overproliferating cells in the tissue, thereby sensitizing the one or more overproliferating cells to a receptor tyrosine kinases inhibitor (I-RTK) and/or amplifying the efficacy of cytotoxic agents in reducing proliferation or viability of the one or more overproliferating cells.

17. The composition of claim 1, further comprising a cytotoxic agent.

18. The composition of claim 17, wherein the cytotoxic agent is cisplatin.

19. A method of treating a proliferative disorder in a subject in need thereof, comprising administering a therapeutically effective amount of the composition of claim 1 to the subject in need thereof

20. The method of claim 19, wherein the composition targets a tissue associated with the proliferative disorder in the subject.

21.-27. (canceled)


SEQUENCE LISTING (12 sequences)

SEQ ID NO: 1 - RNA, 95 nt, Homo sapiens
ccagcucgggcagccguggccaucuuacugggcagcauuggauggagucaggucucuaauacugccugguaaugaugacggcggagcccugcacg

SEQ ID NO: 2 - RNA, 22 nt, Homo sapiens
uaauacugccugguaaugauga

SEQ ID NO: 3 - RNA, 68 nt, Homo sapiens
cccucgucuuacccagcaguguuugggugcgguugggagucucuaauacugccggguaaugauggagg

SEQ ID NO: 4 - RNA, 23 nt, Homo sapiens
uaauacugccggguaaugaugga

SEQ ID NO: 5 - RNA, 83 nt, Homo sapiens
cgccggccgaugggcgucuuaccagacaugguuagaccuggcccucugucuaauacugucugguaaaaccguccauccgcugc

SEQ ID NO: 6 - RNA, 22 nt, Homo sapiens
uaauacugucugguaaaaccgu

SEQ ID NO: 7 - RNA, 95 nt, Homo sapiens
cggccggcccuggguccaucuuccaguacaguguuggauggucuaauugugaagcuccuaacacugucugguaaagauggcucccggguggguuc

SEQ ID NO: 8 - RNA, 22 nt, Homo sapiens
uaacacugucugguaaagaugg

SEQ ID NO: 9 - RNA, 90 nt, Homo sapiens
ccgggccccugugagcaucuuaccggacagugcuggauuucccagcuugacucuaacacugucugguaacgauguucaaaggugacccgc

SEQ ID NO: 10 - RNA, 22 nt, Homo sapiens
uaacacugucugguaacgaugu

SEQ ID NO: 11 - DNA, 21 bp, Artificial Sequence
  Synthetic construct
ccauaaaugcuacgaauautt

SEQ ID NO: 12 - RNA, 21 nt, Artificial Sequence
  Synthetic construct
auauucguagcauuuauggag